(19) United States
(12) Patent Application Publication
(10) Pub. No.: US 2025/0228861 A1
(43) Pub. Date: Jul. 17, 2025

(54) Cancer Treatments Using MTA-Cooperative PRMT5 Inhibitors

(71) Applicant: AMGEN INC., Thousand Oaks, CA (US)

(72) Inventors: Brian Belmontes, Thousand Oaks, CA (US); Edward Lau Yue Chan, Westlake, CA (US); Paul E. Hughes, Ventura, CA (US); Katherine Slemmons, Los Angeles, CA (US); Jan Sun, Camarillo, CA (US)

(21) Appl. No.: 18/853,736

(22) PCT Filed: Apr. 7, 2023
(86) PCT No.: PCT/US2023/017805

§ 371 Date: Oct. 3, 2024

Related U.S. Application Data
(60) Provisional application No. 63/329,010, filed on Apr. 8, 2022.
(60) Provisional application No. 63/345,736, filed on May 25, 2022.

(51) Int. Cl. A61K 31/5377; A61K 31/282; A61K 31/337; A61K 31/4745; A61K 31/506; A61K 31/513; A61K 31/519; A61K 31/7068; A61P 35/00

(52) U.S. Cl. CPC A61K 31/5377; A61K 31/282; A61K 31/337; A61K 31/4745; A61K 31/506; A61K 31/513; A61K 31/519; A61K 31/7068; A61P 35/00

(57) ABSTRACT

Described herein are methods of treating cancer in a patient comprising administering a PRMT5 inhibitor. Also provided are methods of treating cancer in a patient comprising administering a PRMT5 inhibitor and a standard of care therapy.

### BACKGROUND

Epigenetic regulation of gene expression is an important biological determinant of protein production and cellular differentiation and plays a significant pathogenic role in a number of human diseases. Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., PRMT5), many of which are associated with specific genetic alterations that can cause human disease. PRMT5 plays a role in diseases such as proliferative disorders, metabolic disorders, and blood disorders.

The homozygous deletion of tumor suppressor genes is a key driver of cancer, frequently resulting in the collateral loss of passenger genes located in close genomic proximity to the tumor suppressor. Deletion of these passenger genes can create therapeutically tractable vulnerabilities that are specific to tumor cells. Homozygous deletion of the chromosome 9p21 locus, which harbors the well-known tumor suppressor CDKN2A (cyclin dependent kinase inhibitor 2A), occurs in 15% of all tumors and frequently includes the passenger gene MTAP (methylthioadenosine phosphorylase), a key enzyme in the methionine and adenine salvage pathways.

Deletion of MTAP results in accumulation of its substrate, methylthioadenosine (MTA). MTA shares close structural similarity to S-adenosylmethionine (SAM), the substrate methyl donor for the type II methyltransferase PRMT5. Elevated MTA levels, driven by loss of MTAP, selectively compete with SAM for binding to PRMT5, placing the methyltransferase in a hypomorphic state, vulnerable to further PRMT5 inhibition. Multiple genome scale shRNA drop out screens performed in large tumor cell line panels have identified a strong correlation between MTAP loss and cell line dependency on PRMT5, further highlighting the strength of this metabolic vulnerability. However, PRMT5 is a known cell essential gene and conditional PRMT5 knockout and siRNA knockdown studies suggest that significant liabilities could be associated with inhibiting PRMT5 in normal tissues (e.g. pan-cytopenia, infertility, skeletal muscle loss, cardiac hypertrophy, others). Therefore, novel strategies are required to exploit this metabolic vulnerability and preferentially target PRMT5 in MTAP-null tumors while sparing PRMT5 in normal tissues (MTAP WT). Targeting PRMT5 with an MTA-cooperative small molecule inhibitor could preferentially target the MTA bound state of PRMT5, enriched in MTAP-null tumor cells, while providing an improved therapeutic index over normal cells where MTAP is intact and MTA levels are low.

### SUMMARY

In one aspect, described herein is a method of treating cancer in a patient in need thereof comprising administering a PRMT5 inhibitor in an amount ranging from 40 mg to 2000 mg to the patient, wherein the PRMT5 inhibitor comprises a compound of <Formula I> or Compound B or a pharmaceutically acceptable salt thereof:

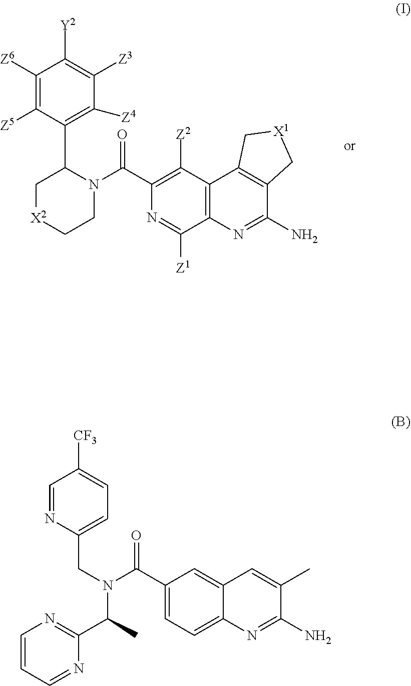

wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride.

In another aspect, described herein is a method of treating cancer in a patient in need thereof comprising administering to the patient
(a) a PRMT5 inhibitor in an amount ranging from 40 mg to 2000 mg, wherein the PRMT5 inhibitor comprises a compound set forth in <Formula 1> or Compound (B) or a pharmaceutically acceptable salt thereof;

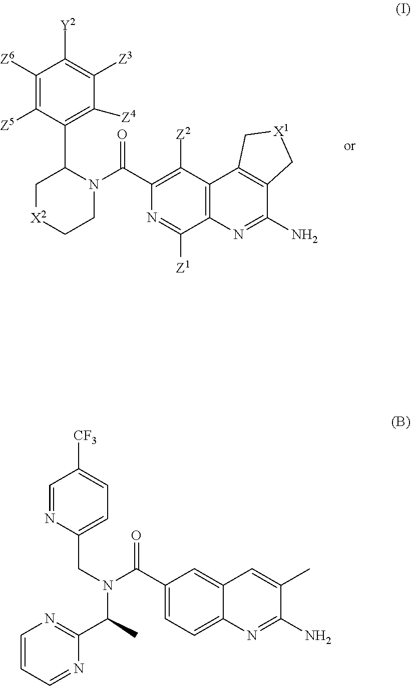

wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride; and
(b) a standard of care therapy for the treatment of cancer.

### BRIEF DESCRIPTION OF THE FIGURES

FIG. 1. Viability (A and B) and SDMA signaling (C and D) in MTAP-null or wild type HAP1 (A/C) and HCT116 (B/D) cells after exposure to Compound B. WT and MTAP-null cells were treated with Compound B or DMSO-only control for 6 days. Viability was measured by CellTiter-Glo and cooperativity was determined as follows: Cooperativity=WT IC50/MTAP-null IC50. (mean±SD, n=3); HAP1 WT and MTAP-null global SDMA levels were assessed by an ELISA assay after 3 days treatment with Compound B; HCT116 WT and MTAP-null global SDMA levels were assessed by an in-cell imaging assay after 4 days treatment with Compound B.

FIG. 2: ELISA analysis of HCT116 MTAP WT and HCT116 MTAP-null contralateral tumor cells. Female Athymic nude mice were dosed with Vehicle or Compound B. Mice were given a total of 4 doses and tumors were collected 4 hours post last dose. Percentage of inhibition reported relative to matched vehicle. Data represent mean±SD, n=3 for each group. STATS: One-Way ANOVA w/Dunnett's Comparison to Control p=0.0213*, p<0.0001****

FIG. 3: Compound B results in significant anti-tumor growth inhibition in endogenous MTAP-null pancreatic (A) and esophageal (B) patient derived xenografts. Female NOD/SCID mice were implanted with PA5415 (pancreatic tumors) or ES11082 (esophageal tumors). Vehicle and Compound B were administered orally (p.o.) once a day for the duration of the study. Data represent group means±SEM, n=10. for each group. B.) STATS: P values were determined by Linear Mixed-Effects Model with a Dunnett's comparison to control; ****p<0.0001

FIG. 4: Tumor growth inhibition in presence of Compound B for endogenous MTAP-null patient derived xenograft models (from left to right) pancreatic; esophageal; esophageal; pancreatic; melanoma; lung; mixed mullerian; lung; melanoma; lung; pancreatic; lung; ovarian; lung; gallbladder; lung; melanoma; melanoma; lung; melanoma; pancreatic; melanoma; pancreatic; brain; and pancreatic. For each model, 6 Female NOD/SCID mice were implanted with PDX. Mean tumor volumes for each grouping were between 100-200 mm³. Mice were allocated to the 2 different study groups by tumor volume and dosing was initiated with Vehicle or Compound B at 100 mg/kg. Plotted data represents the TGI (tumor growth inhibition), n=3 for each group.

FIG. 5: Compound B anti-tumor activity in HCT116 MTAP-null xenografts (A) vs HCT116 wild type xenografts (B). Female Athymic Nude mice were implanted with HCT116 MTAP-null or MTAP WT tumors. Vehicle and Compound B were administered orally (p.o.) once a day for the duration of the study. Data represents group means±SEM, n=10. No effects on body weight observed in either study. STATS: P values were determined by Linear Mixed-Effects Model with a Dunnett's comparison to control. A.) **p<0.01, ****p<0.0001. B.) p=NS.

FIG. 6. Compound B exhibits preferential activity in endogenous MTAP-null tumor cell lines. Waterfall-plot of mean IC50s across a panel of endogenous wild-type (n=11) or MTAP-null (n=15) cell lines treated with the MTA-cooperative PRMT5 inhibitor, Compound B. Cells were seeded for optimal log-phase growth in 96-well plates, incubated overnight, and subsequently treated with compounds across a 3-fold, 9-pt. serial dilution for a 6-day viability assay (Cell-Titer-Glo; Promega). Signal intensity across the dilution series was normalized to DMSO controls (0.1%) and IC50s were calculated utilizing the four-parameter interpolated IC50 model (Prism) averaged across two biological replicates.

FIG. 7 is a graph showing that the combination of Compound G and paclitaxel resulted in significant anti-tumor activity versus either single agent alone H292 NSCLC xenografts.

FIG. 8 is a graph showing that combination of Compound B and paclitaxel results in significant anti-tumor activity versus either single agent alone H292 NSCLC xenografts.

FIG. 9. Compound B preferentially inhibits cell viability in endogenous MTAP-null vs. wildtype (A) DLBCL, (B) pancreatic, and (C) lung cancer cell lines. Representative dose response curves in WT and MTAP-null DLBCL pancreatic and NSCLC cancer tumor cell lines treated with a serial dilution of Compound B for 6 days. Top concentration of tested was 10 μM (1 uM for DLBCL lines) with a total of nine 1:3 serial dilution steps and DMSO-only control. Cell viability was measured by the CellTiter-Glo Luminescence assay. (mean±SD, n=3).

FIG. 10. Compound B exhibits significant anti-tumor activity in DOHH-2 (A and B) and BXPC-3 (C and D) endogenous MTAP-null xenografts. Female SCID mice were implanted with DOHH-2 or BxPC-3 tumors. Vehicle and Compound B were administered orally (p.o.) once a day for the duration of the study. Data represent group means±SEM, n=10. Terminal DOHH-2 or BxPC-3 tumors were collected for SDMA ELISA analysis. Data represent mean±SD, n=5 for each group. STATS: P values were determined by Linear Mixed-Effects Model with a Dunnett's comparison to control; **p<0.01, ****p<0.0001.

FIG. 11. Compound B results in cell cycle arrest and an increase in the DNA damage response in DOHH-2 tumors with no effect on circulating blood cells. (A-D.) Female SCID mice were implanted with DOHH-2 tumors. Once tumors reached 200 mm³ mice were treated with either Vehicle or Compound B at 100 mg/kg. Mice were dosed orally (p.o.) once a day for ten days. Data represent group means±SEM, n=10. A.) Terminal DOHH-2 tumor volumes. B.) Disaggregated Tumors from Brdu (4 hr) pulsed mice treated with vehicle or Compound B treated mice, were co-stained for DAPI and analyzed on flow cytometer. C.) Disaggregated tumors were stained for γH2AX, MFI (mean florescent intensity). D.) Cardiac bleeds were performed and analyzed on ADVIA.

FIG. 12 is a dose matrix showing the Combination Index (CI) scores of Compound B in combination with Paclitaxel.

FIG. 13 is a dose matrix showing the Combination Index (CI) scores of Compound B in combination with Carboplatin.

FIGS. 14A and 14B are graphs showing that the combination of Compound B and paclitaxel (FIG. 14A) and the combination of Compound B and carboplatin (FIG. 14B) results in significant anti-tumor cell growth activity versus either paclitaxel or carboplatin alone in a NSCLC (H292) cell line.

FIGS. 15A and 15B are graphs showing that the combination of Compound B and paclitaxel (FIG. 15A) and the combination of Compound B and carboplatin (FIG. 15B) results in significant anti-tumor activity versus paclitaxel or carboplatin alone in H292 NSCLC xenografts.

### DETAILED DESCRIPTION

The disclosure provides methods of treating cancer in a patient comprising administering a PRMT5 inhibitor, wherein the PRMT5 inhibitor comprises a compound of <Formula I> or Compound (B) or a pharmaceutically acceptable salt thereof:

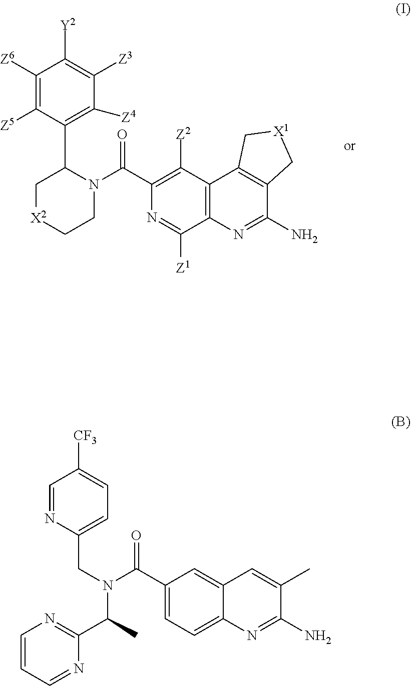

wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride.

In some embodiments, the PRMT5 inhibitor has a structure of Formula (S)-I, or a pharmaceutically acceptable salt thereof:

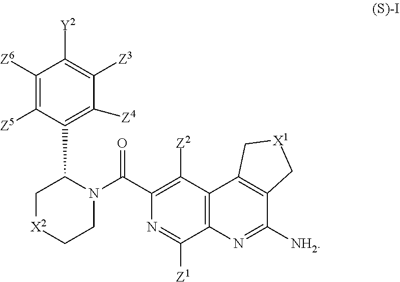

In some embodiments, X¹ is O. In some embodiments, Z¹ and Z² are each H. In some embodiments, X² is O. In some embodiments, each of Z³, Z⁴, Z⁵, and Z⁶ is H. In some embodiments, Y² is C₁-C₆haloalkyl. In some embodiments, Y² is CF₃.

In some cases, the PRMT5 inhibitor is a compound having a structure of: Compound B:

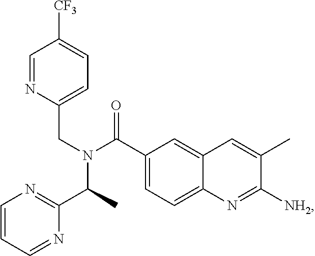 [SMILES: Cc1cc2cc(C(=O)N(Cc3ccc(C(F)(F)F)cn3)[C@@H](C)c3ncccn3)ccc2nc1N]

or salt thereof.

In some embodiments, the PRMT5 inhibitor is a compound having a structure of Compound A:

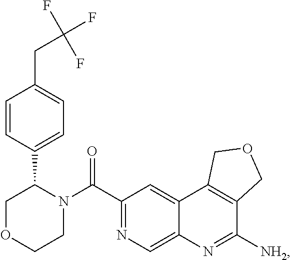 [SMILES: Nc1nc2cnc(C(=O)N3CCOC[C@@H]3c3ccc(CC(F)(F)F)cc3)cc2c2c1COC2]

or salt thereof.

In some embodiments, the PRMT5 inhibitor is compound having a structure of Compound G:

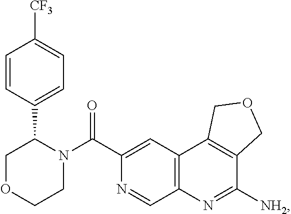 [SMILES: Nc1nc2cnc(C(=O)N3CCOC[C@@H]3c3ccc(C(F)(F)F)cc3)cc2c2c1COC2]

or salt thereof.

Pharmaceutically acceptable salts of the compounds described herein include those derived from suitable inorganic and organic acids and bases.

### Combination Therapy

In some embodiments, the methods further comprise administering a standard of care therapy to the patient as a combination therapy. The term “combination therapy” as used herein refers to the administration of two or more therapeutic agents (e.g., a PRMT5 inhibitor as described herein and a standard of care therapy (e.g., chemotherapy)) to treat cancer. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In some embodiments, the chemotherapy is a platinum-based chemotherapy, i.e., with a platinum agent. Platinum agents (such as carboplatin, oxaliplatin, cisplatin, nedaplatin, satraplatin, lobaplatin, triplatin tetranitrate, picoplatin, ProLindac™ (AP5346), aroplatin, and phenanthriplatin) are widely used antitumor drugs that cause crosslinking of DNA as monoadduct, interstrand crosslinks, intrastrand crosslinks or DNA protein crosslinks.

Carboplatin is a platinum compound alkylating agent that covalently binds to DNA and interferes with DNA function by producing inter-strand DNA cross-links. Platinum-based chemotherapy with or without immunotherapy is a first line regimen for patients with advanced or metastatic non-squamous NSCLC without genomic EGFR or ALK tumor aberrations. Exemplary chemotherapy agent that can be combined with platinum typically include, but are not limited to, pemetrexed, taxanes (paclitaxel, nab-paclitaxel, or docetaxel), and etoposide, or a combination of any of the foregoing.

Carboplatin is a water-soluble platinum complex with the molecular formula of C₆H₁₂N₂O₄Pt and a molecular weight of 373.26. Carboplatin has been assigned the CAS Registration Number 41575-94-4, and is commercially available as PARAPLATIN®, BLASTOCARB®, BLASTOPLATIN®, CARBOKEM®, CARBOMAX®, PARAPLATIN®, CARBOPA®, KARPLAT®, and others. Complete information about carboplatin preparation, dispensing, dosage, and administration schedule can be found in the local package insert (for the United States, see, e.g., CARBOplatin Injection, U.S. Prescribing Information, Fresenius KABI, Lake Zurich, Illinois, 60047 (revision 5/2021), which is herein incorporated by reference in its entirety).

In some embodiments, the chemotherapy is an anti-folate chemotherapeutic agent. In some embodiments, the chemotherapy is pemetrexed or a pharmaceutically acceptable salt thereof. In some embodiments the chemotherapy is premetrexed. In some embodiments, the chemotherapy is pemetrexed disodium. Pemetrexed (N-[4-[2-(2-amino-4,7-dihydro-4-oxo-1H-pyrrolo[2,3-d]pyrimidin-5-yl)ethyl]benzoyl]-L-glutamic acid)) is a folate analog metabolic inhibitor that disrupts folate-dependent metabolic processes essential for cell replication. Pemetrexed is approved by the FDA, in combination with pembrolizumab and platinum chemotherapy, as initial treatment of patients with metastatic non-squamous NSCLC with no EGFR or ALK tumor genomic aberrations. It is also approved in combination with cisplatin for the initial treatment for patients with locally advanced or metastatic non-squamous NSCLC. In the maintenance setting, pemetrexed is approved as a single agent for treatment of patients with locally advanced or metastatic non-squamous NSCLC whose disease has not progressed after four three-week cycles of platinum-based first-line chemotherapy. It is also approved as a single agent for the treatment of patients with recurrent, metastatic non-squamous NSCLC after prior chemotherapy. Pemetrexed is commercially available as ALIMTA®. Complete information about pemetrexed dispensing, dosage and administration schedule can be found in the local package insert (for the United States, see, e.g., ALIMTA® U.S. Prescribing Information, Lilly USA, LLC, Indianapolis, Indiana 46285 (revision 1/2019), which is herein incorporated by reference in its entirety).

In some embodiments, the chemotherapy is a taxane. Exemplary taxanes include, but are not limited to, paclitaxel (TAXOL®); cremophor-free, albumin-engineered nanoparticle formulation of paclitaxel or nab-paclitaxel (ABRAXANE®); and docetaxel (TAXOTERE®).

Paclitaxel is a semisynthetic taxane, a class of anticancer agents that bind to beta tubulin, thereby stabilizing microtubules and inducing cell cycle arrest and apoptosis. Paclitaxel 200 mg/m² administered intravenously over 3 hours every 3 weeks in combination with carboplatin is a standard of care option for the treatment of patients with good performance status, advanced or metastatic, previously untreated NSCLC (Schiller J H et al, 2002). Docetaxel is a semisynthetic taxane, a class of anticancer agents that bind to beta tubulin, thereby stabilizing microtubules and inducing cell-cycle arrest and apoptosis. Docetaxel 75 mg/m² administered intravenously over 1 hour every 3 weeks as a monotherapy is approved by the FDA for the treatment of patients with locally advanced or metastatic NSCLC after failure of prior platinum-based chemotherapy.

Complete information about paclitaxel (TAXOL®) preparation, dispensing, dosage, and administration schedule can be found in the local package insert (for the United States, see, e.g., TAXOL® (paclitaxel) INJECTION U.S. Prescribing Information, Bristol-Myers Squibb Company, Princeton, New Jersey, 08543 (revision 4/2011), which is herein incorporated by reference in its entirety). Complete information about nab-paclitaxel (ABRAXANE®) preparation, dispensing, dosage, and administration schedule can be found in the local package insert (for the United States, see, e.g., ABRAXANE® U.S. Prescribing Information, Bristol-Myers Squibb Company, New Jersey, 08543 (revision 8/2020), which is herein incorporated by reference in its entirety).

Complete information about docetaxel preparation, dispensing, dosage, and administration schedule can be found in the local package insert (for the United States, see, e.g., Docetaxel Injection U.S. Prescribing Information, Sandoz, Princeton, New Jersey, 08540 (revision 3/2012), which is herein incorporated by reference in its entirety).

In some embodiments, the chemotherapeutic agent is docetaxel, paclitaxel, carboplatin, gemcitabine, irinotecan, 5-fluoracil or pemetrexed. In some embodiments, the methods comprise administering carboplatin to the patient. In some embodiments, the methods comprise administering docetaxel to the patient. In some embodiments, the methods comprise administering paclitaxel to the patient. In some embodiments, the methods comprise administering pemetrexed to the patient. In some embodiments, the methods comprise administering gemcitabine to the patient. In some embodiments, the methods comprise administering irinotecan to the patient.

In some embodiments, the methods comprise administering 5-fluoracil to the patient.

### Dosage Regimens

A “therapeutically effective amount” of a PRMT5 inhibitor means an amount effective to treat or to prevent development of, or to alleviate the existing symptoms of, the patient being treated. Determination of the effective amounts is well within the capability of those skilled in the art, especially in light of the detailed disclosure provided herein. Generally, a “therapeutically effective amount” refers to that amount of a PRMT5 inhibitor described herein that results in achieving the desired effect. For example, a therapeutically effective amount of a PRMT5 inhibitor described herein decreases MTAP activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to control.

While individual needs vary, determination of optimal ranges of effective amounts of the compound is within the skill of the art. For administration to a human in the treatment of the conditions and disorders identified herein, for example, typical dosages of a compound as disclosed herein can be about 0.05 mg/kg/day to about 50 mg/kg/day, for example at least 0.05 mg/kg, at least 0.08 mg/kg, at least 0.1 mg/kg, at least 0.2 mg/kg, at least 0.3 mg/kg, at least 0.4 mg/kg, or at least 0.5 mg/kg, such as 50 mg/kg or less, 40 mg/kg or less, 30 mg/kg or less, 20 mg/kg or less, or 10 mg/kg or less, which can be about 2.5 mg/day (0.5 mg/kg×5 kg) to about 5000 mg/day (50 mg/kg×100 kg). For example, dosages of the compound can be about 0.1 mg/kg/day to about 50 mg/kg/day, about 0.05 mg/kg/day to about 10 mg/kg/day, about 0.05 mg/kg/day to about 5 mg/kg/day, about 0.05 mg/kg/day to about 3 mg/kg/day, about 0.07 mg/kg/day to about 3 mg/kg/day, about 0.09 mg/kg/day to about 3 mg/kg/day, about 0.05 mg/kg/day to about 0.1 mg/kg/day, about 0.1 mg/kg/day to about 1 mg/kg/day, about 1 mg/kg/day to about 10 mg/kg/day, about 1 mg/kg/day to about 5 mg/kg/day, about 1 mg/kg/day to about 3 mg/kg/day, about 1 mg/day to about 2000 mg/day, about 20 mg/day to about 1800 mg/day, about 40 mg/day to about 800 mg/day, about 20 mg/day to about 700 mg/day, about 30 mg/day to about 600 mg/day, about 40 mg/day to about 500 mg/day, about 50 mg/day to about 400 mg/day, about 60 mg/day to about 300 mg/day, about 70 mg/day to about 200 mg/day, or about 80 mg/day to about 100 mg/day.

In specific embodiments, a PRMT5 inhibitor described herein is administered to a patient in need thereof orally and once a day. A “patient” or “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

In some embodiments, the patient was treated with a previous line of therapy, i.e., the therapy of the methods disclosed herein is a second (or higher) line of therapy. In some embodiments, the patient was previously treated with chemotherapy prior to treatment with a PRMT5 inhibitor (as disclosed in the methods herein). In some embodiments, the patient was previously treated with a PD1 inhibitor prior to treatment with a PRMT5 inhibitor (as disclosed in the methods herein). In some embodiments, the patient was previously treated with a PDL1 inhibitor prior to treatment with PRMT5 inhibitor (as disclosed in the methods herein).

In some cases, the patient is administered a total daily amount of 40 mg, 120 mg, 240 mg, 480 mg, 800 mg, 960 mg, 1600 mg or 2000 mg of a PRMT5 inhibitor.

In some embodiments, the methods comprise administering a PRMT5 inhibitor described herein in an amount ranging from 40 mg to 2000 mg. In some embodiments, the methods comprise administering 40 mg, 120 mg, 240 mg, 480 mg, 800 mg, 960 mg, 1600 mg, or 2000 mg of the PRMT5 inhibitor to the patient once daily.

In some embodiments, the methods comprise administering docetaxel to the patient. In some embodiments, the methods comprise administering 75 mg/m² docetaxel via IV administration every three weeks.

In some embodiments, the methods described herein comprises administering (a) 40 mg PRMT5 inhibitor daily and (b) 75 mg/m² docetaxel via IV administration every three weeks. In some embodiments, the methods described herein comprises administering (a) 120 mg PRMT5 inhibitor daily and (b) 75 mg/m² docetaxel via IV administration every three weeks. In some embodiments, the methods described herein comprises administering (a) 240 mg PRMT5 inhibitor daily and (b) 75 mg/m² docetaxel via IV administration every three weeks. In some embodiments, the methods described herein comprises administering (a) 480 mg PRMT5 inhibitor daily and (b) 75 mg/m² docetaxel via IV administration every three weeks. In some embodiments, the methods described herein comprises administering (a) 800 mg PRMT5 inhibitor daily and (b) 75 mg/m² docetaxel via IV administration every three weeks. In some embodiments, the methods described herein comprises administering (a) 2000 mg PRMT5 inhibitor daily and (b) 75 mg/m² docetaxel via IV administration every three weeks.

In some embodiments, the methods comprise administering carboplatin to the patient. In some embodiments, the methods comprise administering AUC 5 (or AUC6) carboplatin via IV administration every three weeks. In some embodiments, the methods described herein comprise administering to the patient (a) 40 mg PRMT5 inhibitor daily; (b) AUC 5 (or AUC6) carboplatin via IV administration every three weeks. In some embodiments, the methods described herein comprise administering to the patient (a) 120 mg PRMT5 inhibitor daily; (b) AUC 5 (or AUC6) carboplatin via IV administration every three weeks. In some embodiments, the methods described herein comprise administering to the patient (a) 240 mg PRMT5 inhibitor daily; (b) AUC 5 (or AUC6) carboplatin via IV administration every three weeks. In some embodiments, the methods described herein comprise administering to the patient (a) 480 mg PRMT5 inhibitor daily; (b) AUC 5 (or AUC6) carboplatin via IV administration every three weeks. In some embodiments, the methods described herein comprise administering to the patient (a) 800 mg PRMT5 inhibitor daily; (b) AUC 5 (or AUC6) carboplatin via IV administration every three weeks. In some embodiments, the methods described herein comprise administering to the patient (a) 2000 mg PRMT5 inhibitor daily; (b) AUC 5 (or AUC6) carboplatin via IV administration every three weeks.

In some embodiments, the methods comprise administering paclitaxel to the patient. In some embodiments, the methods comprise administering 100 mg/m² paclitaxel via IV administration weekly (or 135 mg/m² paclitaxel via IV administration every three weeks). In some embodiments, the methods comprise administering to the patient (a) 40 mg PRMT5 inhibitor daily; and (b) administering 100 mg/m² paclitaxel via IV administration weekly (or 135 mg/m² paclitaxel via IV administration every three weeks). In some embodiments, the methods comprise administering to the patient (a) 80 mg PRMT5 inhibitor daily; and (b) administering 100 mg/m² paclitaxel via IV administration weekly (or 135 mg/m² paclitaxel via IV administration every three weeks).

In some embodiments, the methods comprise administering to the patient (a) 120 mg PRMT5 inhibitor daily; and (b) administering 100 mg/m² paclitaxel via IV administration weekly (or 135 mg/m² paclitaxel via IV administration every three weeks). In some embodiments, the methods comprise administering to the patient (a) 240 mg PRMT5 inhibitor daily; and (b) administering 100 mg/m² paclitaxel via IV administration weekly (or 135 mg/m² paclitaxel via IV administration every three weeks). In some embodiments, the methods comprise administering to the patient (a) 800 mg PRMT5 inhibitor daily; and (b) administering 100 mg/m² paclitaxel via IV administration weekly (or 135 mg/m² paclitaxel via IV administration every three weeks). In some embodiments, the methods comprise administering to the patient (a) 2000 mg PRMT5 inhibitor daily; and (b) administering 100 mg/m² paclitaxel via IV administration weekly (or 135 mg/m² paclitaxel via IV administration every three weeks).

In some embodiments, the methods comprise administering pemetrexed to the patient. In some embodiments, the methods comprise administering 500 mg/m² pemetrexed via IV administration every three weeks. In some embodiments, the methods described herein comprise administering to the patient (a) 40 mg PRMT5 inhibitor daily; and (b) 500 mg/m² pemetrexed via IV administration every three weeks. In some embodiments, the methods described herein comprise administering to the patient (a) 80 mg PRMT5 inhibitor daily; and (b) 500 mg/m² pemetrexed via IV administration every three weeks. In some embodiments, the methods described herein comprise administering to the patient (a) 120 mg PRMT5 inhibitor daily; and (b) 500 mg/m² pemetrexed via IV administration every three weeks. In some embodiments, the methods described herein comprise administering to the patient (a) 240 mg PRMT5 inhibitor daily; and (b) 500 mg/m² pemetrexed via IV administration every three weeks. In some embodiments, the methods described herein comprise administering to the patient (a) 480 mg PRMT5 inhibitor daily; and (b) 500 mg/m² pemetrexed via IV administration every three weeks. In some embodiments, the methods described herein comprise administering to the patient (a) 800 mg PRMT5 inhibitor daily; and (b) 500 mg/m² pemetrexed via IV administration every three weeks. In some embodiments, the methods described herein comprise administering to the patient (a) 2000 mg PRMT5 inhibitor daily; and (b) 500 mg/m² pemetrexed via IV administration every three weeks.

In some embodiments, the methods comprise administering gemcitabine to the patient. In some embodiments, the methods comprise administering 1000 mg/m² gemcitabine as an intravenous infusion over 30 minutes on Days 1, 8 and 15 of each 28-day cycle. In some embodiments, the methods described herein comprise administering to the patient (a) 40 mg PRMT5 inhibitor daily; and (b) 1000 mg/m² gemcitabine as an intravenous infusion over 30 minutes on Days 1, 8 and 15 of each 28-day cycle. In some embodiments, the methods described herein comprise administering to the patient (a) 80 mg PRMT5 inhibitor daily; and (b) 1000 mg/m² gemcitabine as an intravenous infusion over 30 minutes on Days 1, 8 and 15 of each 28-day cycle. In some embodiments, the methods described herein comprise administering to the patient (a) 120 mg PRMT5 inhibitor daily; and (b) 1000 mg/m² gemcitabine as an intravenous infusion over 30 minutes on Days 1, 8 and 15 of each 28-day cycle. In some embodiments, the methods described herein comprise administering to the patient (a) 480 mg PRMT5 inhibitor daily; and (b) 1000 mg/m² gemcitabine as an intravenous infusion over 30 minutes on Days 1, 8 and 15 of each 28-day cycle. In some embodiments, the methods described herein comprise administering to the patient (a) 800 mg PRMT5 inhibitor daily; and (b) 1000 mg/m² gemcitabine as an intravenous infusion over 30 minutes on Days 1, 8 and 15 of each 28-day cycle. In some embodiments, the methods described herein comprise administering to the patient (a) 2000 mg PRMT5 inhibitor daily; and (b) 1000 mg/m² gemcitabine as an intravenous infusion over 30 minutes on Days 1, 8 and 15 of each 28-day cycle.

In some embodiments, the methods comprise administering 1250 mg/m² gemcitabine as an intravenous infusion over 30 minutes on Days 1 and 8 of each 21-day cycle. In some embodiments, the methods described herein comprise administering to the patient (a) 40 mg PRMT5 inhibitor daily; and (b) 1250 mg/m² gemcitabine via IV infusion over 30 minutes on Days 1 and 8 of each 21-day cycle. In some embodiments, the methods described herein comprise administering to the patient (a) 80 mg PRMT5 inhibitor daily; and (b) 1250 mg/m² gemcitabine via IV infusion over 30 minutes on Days 1 and 8 of each 21-day cycle. In some embodiments, the methods described herein comprise administering to the patient (a) 120 mg PRMT5 inhibitor daily; and (b) 1250 mg/m² gemcitabine via IV infusion over 30 minutes on Days 1 and 8 of each 21-day cycle. In some embodiments, the methods described herein comprise administering to the patient (a) 480 mg PRMT5 inhibitor daily; and (b) 1250 mg/m² gemcitabine via IV infusion over 30 minutes on Days 1 and 8 of each 21-day cycle. In some embodiments, the methods described herein comprise administering to the patient (a) 800 mg PRMT5 inhibitor daily; and (b) 1250 mg/m² gemcitabine via IV infusion over 30 minutes on Days 1 and 8 of each 21-day cycle. In some embodiments, the methods described herein comprise administering to the patient (a) 2000 mg PRMT5 inhibitor daily; and (b) 1250 mg/m² gemcitabine via IV infusion over 30 minutes on Days 1 and 8 of each 21-day cycle.

In some embodiments, the methods comprise administering irinotecan to the patient. In some embodiments, the methods comprise administering 180 mg/m² irinotecan via IV administration every two weeks. In some embodiments, the methods described herein comprise administering to the patient (a) 40 mg PRMT5 inhibitor daily; and (b) 180 mg/m² irinotecan via IV administration every two weeks. In some embodiments, the methods described herein comprise administering to the patient (a) 80 mg PRMT5 inhibitor daily; and (b) 180 mg/m² irinotecan via IV administration every two weeks. In some embodiments, the methods described herein comprise administering to the patient (a) 120 mg PRMT5 inhibitor daily; and (b) 180 mg/m² irinotecan via IV administration every two weeks. In some embodiments, the methods described herein comprise administering to the patient (a) 480 mg PRMT5 inhibitor daily; and (b) 180 mg/m² irinotecan via IV administration every two weeks. In some embodiments, the methods described herein comprise administering to the patient (a) 800 mg PRMT5 inhibitor daily; and (b) 180 mg/m² irinotecan via IV administration every two weeks. In some embodiments, the methods described herein comprise administering to the patient (a) 2000 mg PRMT5 inhibitor daily; and (b) 180 mg/m² irinotecan via IV administration every two weeks.

In some embodiments, the methods comprise administering 150 mg/m² irinotecan via IV administration every two weeks. In some embodiments, the methods described herein comprise administering to the patient (a) 40 mg PRMT5 inhibitor daily; and (b) 150 mg/m² irinotecan via IV administration every two weeks. In some embodiments, the methods described herein comprise administering to the patient (a) 80 mg PRMT5 inhibitor daily; and (b) 150 mg/m² irinotecan via IV administration every two weeks. In some embodiments, the methods described herein comprise administering to the patient (a) 120 mg PRMT5 inhibitor daily; and (b) 150 mg/m² irinotecan via IV administration every two weeks. In some embodiments, the methods described herein comprise administering to the patient (a) 480 mg PRMT5 inhibitor daily; and (b) 150 mg/m² irinotecan via IV administration every two weeks. In some embodiments, the methods described herein comprise administering to the patient (a) 800 mg PRMT5 inhibitor daily; and (b) 150 mg/m² irinotecan via IV administration every two weeks. In some embodiments, the methods described herein comprise administering to the patient (a) 2000 mg PRMT5 inhibitor daily; and (b) 150 mg/m² irinotecan via IV administration every two weeks.

In some embodiments, the methods comprise administering 5-fluoracil to the patient. In some embodiments, the methods comprise administering 400 mg/m² 5-fluoracil IV bolus then 2400-3000 mg/m² IV continuous infusion×46 hrs. In some embodiments, the methods described herein comprise administering to the patient (a) 40 mg PRMT5 inhibitor daily; and (b) 400 mg/m² 5-fluoracil IV bolus then 2400-3000 mg/m² IV continuous infusion×46 hrs. In some embodiments, the methods described herein comprise administering to the patient (a) 80 mg PRMT5 inhibitor daily; and (b) 400 mg/m² 5-fluoracil IV bolus then 2400-3000 mg/m² IV continuous infusion×46 hrs. In some embodiments, the methods described herein comprise administering to the patient (a) 120 mg PRMT5 inhibitor daily; and (b) 400 mg/m² 5-fluoracil IV bolus then 2400-3000 mg/m² IV continuous infusion×46 hrs. In some embodiments, the methods described herein comprise administering to the patient (a) 240 mg PRMT5 inhibitor daily; and (b) 400 mg/m² 5-fluoracil IV bolus then 2400-3000 mg/m² IV continuous infusion×46 hrs. In some embodiments, the methods described herein comprise administering to the patient (a) 800 mg PRMT5 inhibitor daily; and (b) 400 mg/m² 5-fluoracil IV bolus then 2400-3000 mg/m² IV continuous infusion×46 hrs. In some embodiments, the methods described herein comprise administering to the patient (a) 2000 mg PRMT5 inhibitor daily; and (b) 400 mg/m² 5-fluoracil IV bolus then 2400-3000 mg/m² IV continuous infusion×46 hrs.

In some embodiments, the methods comprise administering 5-fluoracil to the patient. In some embodiments, the methods comprise administering 1200 mg/m² 5-fluoracil via IV continuous infusion×44 hrs. In some embodiments, the methods described herein comprise administering to the patient (a) 40 mg PRMT5 inhibitor daily; and (b) 1200 mg/m² 5-fluoracil via IV continuous infusion×44 hrs. In some embodiments, the methods described herein comprise administering to the patient (a) 80 mg PRMT5 inhibitor daily; and (b) 1200 mg/m² 5-fluoracil via IV continuous infusion×44 hrs. In some embodiments, the methods described herein comprise administering to the patient (a) 120 mg PRMT5 inhibitor daily; and (b) 1200 mg/m² 5-fluoracil via IV continuous infusion×44 hrs. In some embodiments, the methods described herein comprise administering to the patient (a) 480 mg PRMT5 inhibitor daily; and (b) 1200 mg/m² 5-fluoracil via IV continuous infusion×44 hrs. In some embodiments, the methods described herein comprise administering to the patient (a) 800 mg PRMT5 inhibitor daily; and (b) 1200 mg/m² 5-fluoracil via IV continuous infusion×44 hrs. In some embodiments, the methods described herein comprise administering to the patient (a) 2000 mg PRMT5 inhibitor daily; and (b) 1200 mg/m² 5-fluoracil via IV continuous infusion×44 hrs.

### Cancer

In some embodiments, the cancer is an MTAP-deficient and/or MTA-accumulating cancer. An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In a patient afflicted with a MTAP-deficiency-related disease, it is possible that some disease cells (e.g., cancer cells) can be MTAP-deficient while others are not.

Similarly, some disease cells may be MTA-accumulating while others are not. Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor. Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

In some embodiments, the cancer is acute myeloid leukemia, cancer in adolescents, childhood adrenocortical carcinoma childhood, AIDS-related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some cases, the cancer is pancreatic cancer; esophageal cancer; melanoma; lung cancer; mixed mullerian cancer; ovarian cancer; or gallbladder cancer.

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Pharmaceutical Formulations and Routes of Administration

Pharmaceutical compositions containing a PRMT5 inhibitor described herein can be manufactured in a conventional manner, e.g., by conventional mixing, dissolving, granulating, dragee-making, levigating, emulsifying, encapsulating, entrapping, or lyophilizing processes. Proper formulation is dependent upon the route of administration chosen.

### Monitoring Efficacy of Treatment

The efficacy of a given treatment for cancer can be determined by the skilled clinician. However, a treatment is considered “effective treatment,” as the term is used herein, if any one or all of the signs or symptoms of e.g., a tumor are altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application.

### EMBODIMENTS

1. A method of treating cancer in a patient in need thereof comprising administering a PRMT5 inhibitor in an amount ranging from 40 mg to 2000 mg to the patient, wherein the PRMT5 inhibitor comprises a compound of <Formula 1> or Compound (B) or a pharmaceutically acceptable salt thereof:

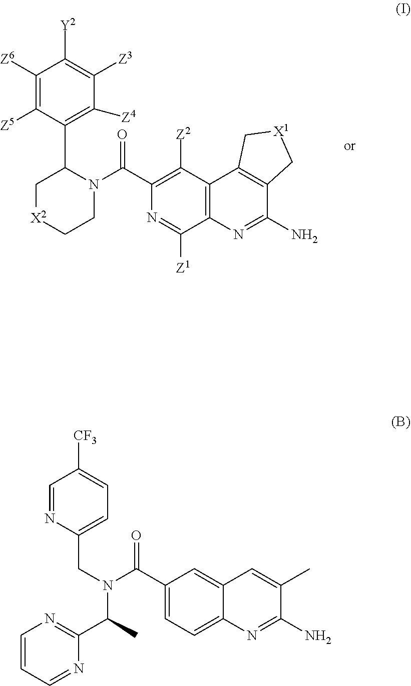

wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride.
2. The method of embodiment 1, wherein the cancer is a MTAP-null cancer.
3. The method of embodiment 2, wherein the MTAP-null cancer is glioblastoma, mesothelioma, soft tissue sarcoma, esophageal cancer, melanoma, lymphoma/leukemia, head and neck cancer, cholangiocarcinoma, stomach cancer, glioma, thymoma, adenocystic carcinoma, pancreatic cancer, lung cancer, breast cancer, liver cancer or bladder cancer.
4. The method of embodiment 3, wherein the lung cancer is non-squamous cell lung cancer (NSCLC).
5. The method of any one of embodiments 1-4, wherein the PRMT5 inhibitor has a structure of Formula (S)-1, or a pharmaceutically acceptable salt thereof:

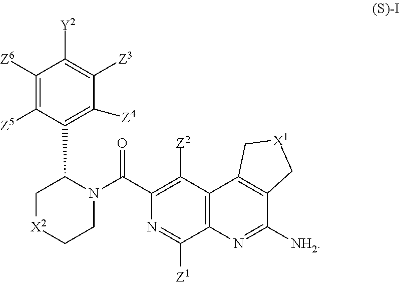

6. The method of any one of embodiments, 1-5, wherein:
   X¹ is O;
   Z¹ and Z² are each H;
   X² is O;
   Z³, Z⁴, Z⁵, and Z⁶ are each H;
   Y² is C₁-C₆haloalkyl; and
   Y² is CF₃.
7. The method of any one of embodiments 1-6, further comprising administering paclitaxel to the subject.
8. The method of embodiment 7, comprising administering to the subject
   (a) 40-2000 mg of the PRMT5 inhibitor; and
   (b) 75 mg/m² paclitaxel.
9. The method of embodiment 7, comprising administering to the subject
   (a) 40-2000 mg of the PRMT5 inhibitor; and
   (b) 100 mg/m² paclitaxel.
10. The method of embodiment 7, comprising administering to the subject
   (a) 40-2000 mg of the PRMT5 inhibitor; and
   (b) 135 mg/m² paclitaxel.
11. The method of any one of embodiments 1-6, further comprising administering carboplatin to the subject.
12. The method of embodiment 11, comprising administering to the subject
   (a) 40-2000 mg of the PRMT5 inhibitor; and
   (b) AUC5-AUC6 carboplatin.
13. The method of any one of claims 1-6, further comprising administering gemcitabine to the subject.
14. The method of embodiment 13, comprising administering to the subject
   (a) 40-2000 mg of the PRMT5 inhibitor; and
   (b) 1000 mg/m²-1250 mg/m² gemcitabine.
15. The method of any one of claims 1-6, further comprising administering irinotecan to the subject.
16. The method of embodiment 15, comprising administering to the subject
   (a) 40-2000 mg of the PRMT5 inhibitor; and
   (b) 150 mg/m²-180 mg/m² irinotecan.
17. The method of any one of embodiments 1-6, further comprising administering 5-fluoracil to the subject.
18. The method of embodiment 17, comprising administering to the subject
   (a) 40-2000 mg of the PRMT5 inhibitor; and
   (b) 400 mg/m²-1200 mg/m² 5-fluoracil.
19. The method of any one of embodiments 1-6, further comprising administering pemetrexed to the subject.
20. The method of embodiment 19, comprising administering to the subject
   (a) 40-2000 mg of the PRMT5 inhibitor; and
   (b) 500 mg/m² pemetrexed.
21. The method of any one of embodiments 1-20, wherein the PRMT5 inhibitor comprises a compound having a structure of Compound G:

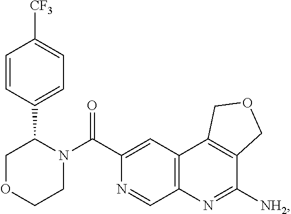 [SMILES: Nc1nc2cnc(C(=O)N3CCOC[C@@H]3c3ccc(C(F)(F)F)cc3)cc2c2c1COC2]

or salt thereof.
22. The method of any one of embodiments 1-20, wherein the PRMT5 inhibitor comprises Compound B or salt thereof.
23. The method of any one of embodiments 1-20, wherein the PRMT5 inhibitor comprises a compound having a structure of Compound A:

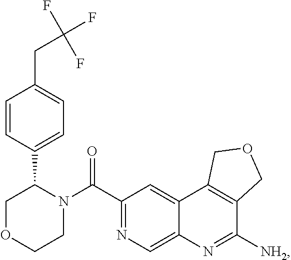 [SMILES: Nc1nc2cnc(C(=O)N3CCOC[C@@H]3c3ccc(CC(F)(F)F)cc3)cc2c2c1COC2]

or salt thereof.
24. A method of treating cancer in a patient in need thereof comprising administering to the patient
(a) a PRMT5 inhibitor in an amount ranging from 40 mg to 2000 mg, wherein the PRMT5 inhibitor comprises a compound set forth in <Formula 1> or Compound B, or a pharmaceutically acceptable salt thereof;

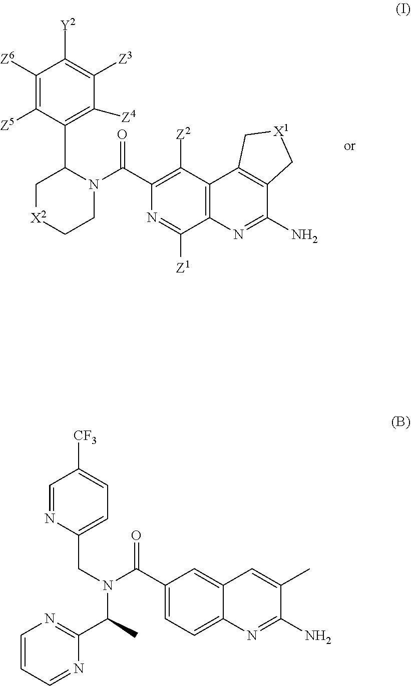

wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride; and
(b) a standard of care therapy for the treatment of cancer.
25. The method of embodiment 24, wherein the standard of care therapy comprises chemotherapy.
26. The method of embodiment 25, wherein the chemotherapy comprises paclitaxel, carboplatin, gemcitabine, irinotecan, 5-fluoracil or pemetrexed or a combination thereof.
27. The method of any one of embodiments 24-26, wherein the PRMT5 inhibitor has a structure of Formula (S)-I, or a pharmaceutically acceptable salt thereof:

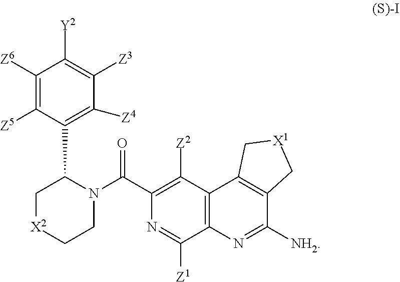

28. The method of any one of embodiments 24-27, wherein
   X¹ is O;
   Z¹ and Z² are each H;
   X² is O;
   Z³, Z⁴, Z⁵, and Z⁶ are each H;
   Y² is C₁-C₆haloalkyl; and
   Y² is CF₃.
29. The method of any one of embodiments 25-28, wherein the chemotherapy comprises paclitaxel.
30. The method of embodiments 29, comprising administering to the subject
   (a) 40-2000 mg of the PRMT5 inhibitor; and
   (b) 75 mg/m²-135 mg/m² paclitaxel.
31. The method of any one of embodiments 25-28, wherein the chemotherapy comprises carboplatin.
32. The method of embodiment 31, comprising administering to the subject
   (a) 40-2000 mg of the PRMT5 inhibitor; and
   (b) AUC5-AUC6 carboplatin.
33. The method of any one of embodiments 25-28, wherein the chemotherapy comprises gemcitabine.
34. The method of embodiment 33, comprising administering to the subject
   (a) 40-2000 mg of the PRMT5 inhibitor; and
   (b) 1000 mg/m²-1250 mg/m² gemcitabine.
35. The method of any one of embodiments 25-28, wherein the chemotherapy comprises irinotecan.
36. The method of embodiment 35, comprising administering to the subject
   (a) 40-2000 mg of the PRMT5 inhibitor; and
   (b) 150 mg/m²-180 mg/m² irinotecan.
37. The method of any one of embodiments 25-28, wherein the chemotherapy comprises 5-fluoracil.
38. The method of embodiment 37, comprising administering to the subject
   (a) 40-800 mg of the PRMT5 inhibitor; and
   (b) 400 mg/m²-1200 mg/m² 5-fluoracil.
39. The method of any one of embodiments 25-28, wherein the chemotherapy comprises pemetrexed.
40. The method of embodiment 39, comprising administering to the subject
   (a) 40-2000 mg of the PRMT5 inhibitor; and
   (b) 500 mg/m² pemetrexed.
41. The method of any one of embodiments 24-40, wherein the PRMT5 inhibitor comprises a compound having a structure of Compound G:

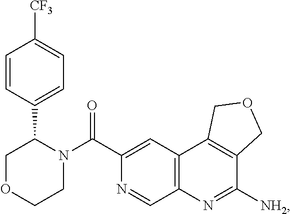 [SMILES: Nc1nc2cnc(C(=O)N3CCOC[C@@H]3c3ccc(C(F)(F)F)cc3)cc2c2c1COC2]

or salt thereof.
42. The method of any one of embodiments 24-40, wherein the PRMT5 inhibitor comprises Compound B, or salt thereof.
43. The method of any one of embodiments 24-40, wherein the PRMT5 inhibitor is a compound having a structure of Compound A:

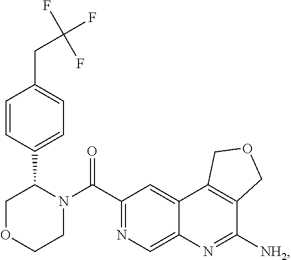 [SMILES: Nc1nc2cnc(C(=O)N3CCOC[C@@H]3c3ccc(CC(F)(F)F)cc3)cc2c2c1COC2]

or salt thereof.
44. The method of any one of embodiments 24-43, wherein the PRMT5 inhibitor and the standard of care therapy are administered concurrently.
45. The method of any one of embodiments 24-43, wherein the PRMT5 inhibitor and the standard of care therapy are administered sequentially.

### EXAMPLES

### Example 1—Combination of a Methylthioadenosine (MTA)-Cooperative PRMT5 Inhibitor and Chemotherapy in Patients with Advanced Methylthioadenosine Phosphorylase (MTAP)-Null Solid Tumors

Compound G is an MTA-cooperative PRMT5i that preferentially targets the MTA-bound state of PRMT5 that is enriched in MTAP-null tumors and thus represents a novel strategy to enhance the therapeutic margin of this class of inhibitors.

Eligible patients (≥18 years) with histologically confirmed locally advanced/metastatic STs not amenable to surgery and/or radiation, homozygous MTAP and/or CDKN2A deletion (by local next generation sequencing), MTAP protein loss in ST (by central immunohistochemistry), measurable disease, ECOG PS 0-1, adequate hematopoietic, renal, liver, pulmonary, cardiac, coagulation function and glucose control will be included. The study has 3 parts, each with subparts. Here, we describe Parts 1c-h (Compound G dose expansion) and 2 (Compound G+docetaxel dose exploration [a] and expansion [b]) in patients with squamous non-small cell lung cancer (NSCLC)(1c), adeno-NSCLC (1d), cholangiocarcinoma (1e), head and neck squamous cell carcinoma (1f), pancreatic adenocarcinoma (1g), other STs excluding primary brain tumor, and lymphoma (1h), and NSCLC (2a/b). The primary endpoints include dose-limiting toxicities, adverse events, ECGs, lab abnormalities, and vital signs. Secondary endpoints include Cmax, Tmax, and AUC after single or multiple doses, time to response, stable disease, progression-free survival, overall survival, objective response, disease control, and duration of response. This study will enroll ˜290 and 50 pts in Parts 1 and 2, respectively

The clinical study above will be repeated with Compound G in combination with several other chemotherapeutic agents (e.g., paclitaxel, carboplatin, gemcitabine, irinotecan, 5-fluoracil or pemetrexed).

### Example 2—Effect of PRMT5 Inhibitors in MTAP-Null Cell Lines

The following Example assessed the effect of PRMT5 inhibitors (e.g., Compound B and Compound G) in MTAP-null (HTAP and H116) cell lines. Compound A is assessed using the same protocol.

Plated cells were at optimized cell density in 96-well black walled, clear bottom tissue culture plates in 90 μL of growth media. Cells were incubated at room temperature for 30 minutes prior to placing in incubator at 37° C. and 5% CO₂ overnight. The next day, PRNT5 inhibitors (e.g., Compound B and Compound G) were diluted serially in DMSO with a 1:3 dilution factor in 96-well V-Bottom plates. Secondary compound dilutions (1:100) in media were done in the 96-well V-Bottom plate by adding 3 μL of the primary dilution to 297 μL of culture media. Finally, 10 μL from the secondary compound dilutions were added to cells in triplicate (1:10 dilution; final DMSO concentration 0.1%). Once addition was complete, plates were incubated at 37° C. and 5% CO₂. Cell viability was measured by the CellTiter-Glo Luminescence assay after 6 days of treatment. Percent of Control (POC) values were calculated as follows: POC=100*(Treatment/Vehicle). Mean POC values were then calculated for each treatment condition and used to fit dose response curves applying a four parameter logistic curve in GraphPad Prism 7.

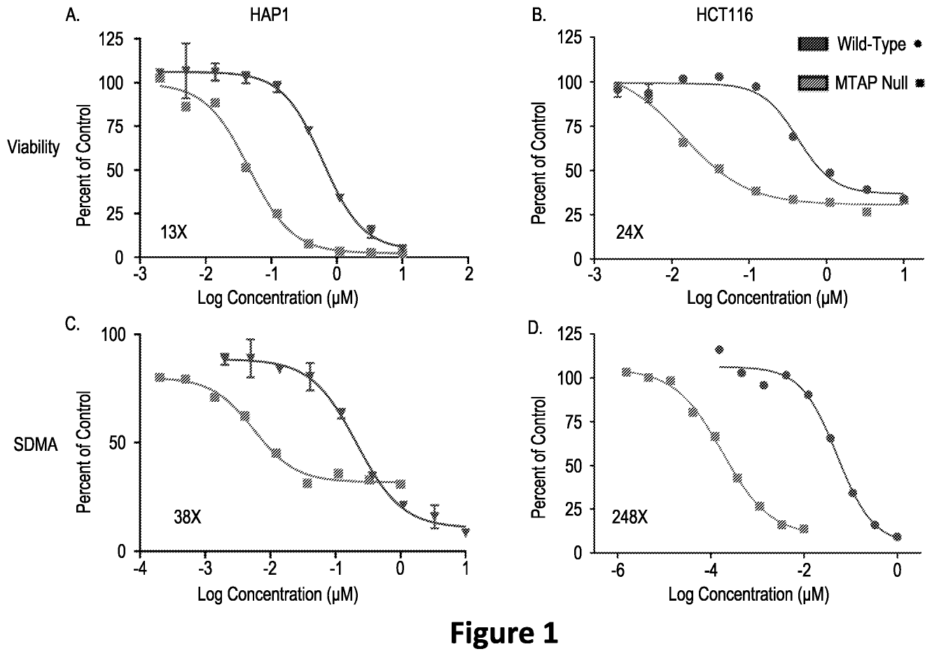

HAP1 WT and MTAP-null global SDMA levels were assessed by an ELISA assay after 3 days treatment. Results are shown in FIG. 1C.

HCT116 WT and MTAP-null global SDMA levels were assessed by an in-cell imaging assay after 4 days treatment. Results are shown in FIG. 1D.

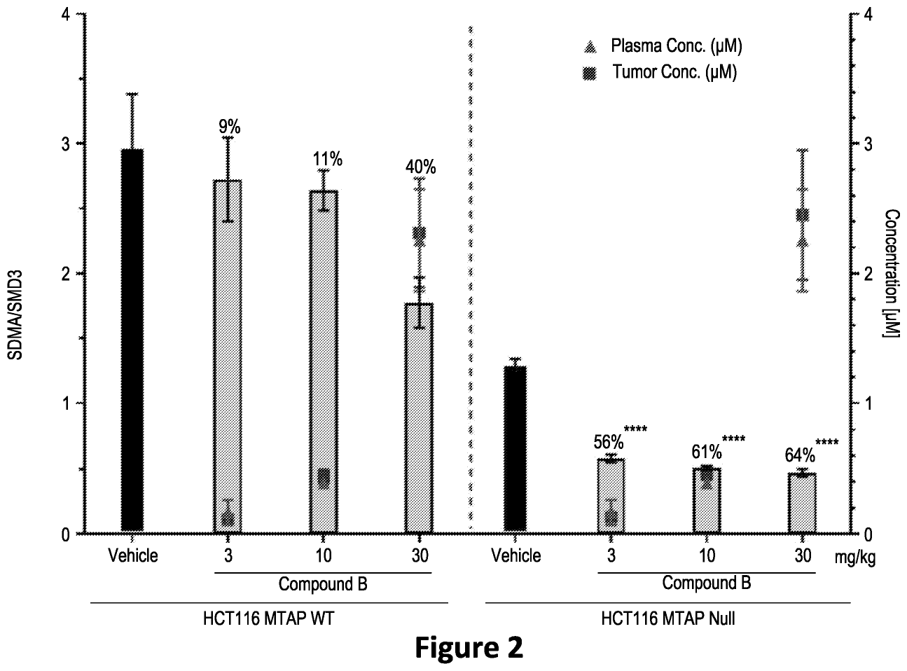

Following treatment with Compound B, the levels of SDMA were lower in both HAP1- and HCT116 MTAP-null cells (IC50=0.0002 μM) compared to HCT116 MTAP-WT cells (IC50=0.050 μM). See FIGS. 1C and 1D. Compound B also selectively inhibited the proliferation of both HAP1- and HCT116 MTAP-null cells (IC50=0.027 μM) compared to HCT116 MTAP-WT cells (IC50=0.63 μM). See FIGS. 1A and 1B. The profiling of Compound B in an expanded panel of tumor cell lines demonstrated that Compound B inhibited the proliferation of most MTAP-null cells, with minimal effects on MTAP-WT cells. Finally, in-vitro mechanism of action studies demonstrated that treatment with Compound B induced DNA damage, as illustrated by increased phosphorylation of H2AX, and an arrest in the G2/M phase of the cell cycle in MTAP-null cells (data not shown). In vivo, oral administration of Compound B selectively inhibits SDMA and tumor growth in HCT116 MTAP-null tumor xenografts, compared to the HCT116 MTAP-WT xenografts. See FIG. 2.

### Example 3—Combination of PRMT5 Inhibitor and Paclitaxel in NSCLC Cell Lines

NSCLC cell lines (H292 A549) were treated with the combination of a PRMT5 inhibitor (i.e., Compound B or Compound G) and paclitaxel for 6 days. A PRMT5 inhibitor (i.e., Compound B or Compound G) was performed at a 1.9-fold dilution series and the combination partner was performed at 1.2 to 1.7 fold dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{T r e a t m e n t}{Average of vehicle} )$$

Synergy analysis was performed using the CalcuSyn software to determine CI scores based on the drug concentrations used and corresponding Fa values. Results are shown Tables 1-4 below. *CI Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1.

TABLE 1
Representative Compound G and paclitaxel concentrations and
corresponding combination Fa and CI scores in H292 cells.
H292 Combination Index (CI) Determination

| Dose, Fixed | Dose, Variable | Combination | Computed |
|---|---|---|---|
| Compound G (μM) | Paclitaxel (μM) | Fa | CI value* |
| 0.437382 | 0.0025 | 0.974 | 0.94 |
| 0.437382 | 0.0019 | 0.957 | 0.85 |
| 0.437382 | 0.0015 | 0.9417 | 0.749 |
| 0.437382 | 0.0011 | 0.8929 | 0.704 |
| 0.437382 | 0.0009 | 0.8617 | 0.658 |
| 0.437382 | 0.0007 | 0.7749 | 0.728 |
| 0.437382 | 0.0005 | 0.6656 | 0.92 |

TABLE 2
Representative Compound B and paclitaxel concentrations
and corresponding combination Fa and CI scores in H292
cells. CalcuSyn software was used to generate CI scores.
H292 Combination Index (CI) Determination

| Dose, Fixed | Dose, Variable | Combination | Computed |
|---|---|---|---|
| Compound B (μM) | Paclitaxel (μM) | Fa | CI value* |
| 0.2916 | 0.0025 | 0.9674 | 0.928 |
| 0.2916 | 0.0019 | 0.9568 | 0.8 |
| 0.2916 | 0.0015 | 0.9425 | 0.697 |
| 0.2916 | 0.0011 | 0.8932 | 0.724 |
| 0.2916 | 0.0009 | 0.8302 | 0.757 |
| 0.2916 | 0.0007 | 0.7796 | 0.758 |
| 0.2916 | 0.0005 | 0.7127 | 0.84 |

TABLE 3
Representative Compound G and paclitaxel concentrations and
corresponding combination Fa and CI scores in A-549 cells.
A549 Combination Index (CI) Determination

| Dose, Fixed | Dose, Variable | Combination | Computed |
|---|---|---|---|
| Compound G (μM) | Paclitaxel (μM) | Fa | CI value* |
| 0.437382 | 0.0025 | 0.9687 | 0.869 |
| 0.437382 | 0.0019 | 0.95 | 0.759 |
| 0.437382 | 0.0015 | 0.9048 | 0.736 |
| 0.437382 | 0.0011 | 0.7817 | 0.77 |
| 0.437382 | 0.0009 | 0.7048 | 0.786 |
| 0.437382 | 0.0007 | 0.6087 | 0.863 |
| 0.437382 | 0.0005 | 0.6362 | 0.639 |

TABLE 4
Representative Compound B and paclitaxel concentrations and
corresponding combination Fa and CI scores in A-549 cells.
A549 Combination Index (CI) Determination

| Dose, Fixed | Dose, Variable | Combination | Computed |
|---|---|---|---|
| Compound B (μM) | Paclitaxel (μM) | Fa | CI value* |
| 0.1535 | 0.0025 | 0.9692 | 0.898 |
| 0.1535 | 0.0019 | 0.9387 | 0.867 |
| 0.1535 | 0.0015 | 0.9023 | 0.786 |
| 0.1535 | 0.0011 | 0.8365 | 0.745 |
| 0.1535 | 0.0009 | 0.7578 | 0.716 |
| 0.1535 | 0.0007 | 0.7177 | 0.64 |
| 0.1535 | 0.0005 | 0.7030 | 0.545 |

As shown in Tables 1-4, most of the CI scores are in the moderate synergism and slightly synergism range.

### Example 4—Combination of PRMT5 Inhibitor and Paclitaxel in NSCLC Cell Lines

NSCLC cell lines (H292, A549) are treated with the combination of a PRMT5 inhibitor (i.e., Compound A) and paclitaxel for 6 days. PRMT5 inhibitor (Compound A) is performed at a 1.9-fold dilution series and the combination partner is performed at 1.2 to 1.7 fold dilution series to create an 8×10 dose matrix including DMSO-only controls. Cell viability is measured by the CellTiter-Glo Luminescence assay.

### Example 5—Combination of PRMT5 Inhibitor and Gemcitabine in Pancreatic Cell Lines

Pancreatic cancer cell lines (MIAPACA2T2, PSN1) were treated with the combination of a PRMT5 inhibitor (i.e., Compound B or Compound G) and gemcitabine for 6 days. A PRMT5 inhibitor (i.e., Compound B or Compound G) was performed at a 1.9-fold dilution series and the combination partner was performed at 1.2 to 1.7 fold dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{T r e a t m e n t}{Average of vehicle} )$$

Synergy analysis was performed using the CalcuSyn software to determine CI scores based on the drug concentrations used and corresponding Fa values. Results are shown Tables 5-8 below. *C Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1.

TABLE 5
Representative Compound G and Gemcitabine concentrations and
corresponding combination Fa and CI scores in MIAPACA2T2 cells.
MIAPACA2T2 Combination Index (CI) Determination

| Dose, Fixed | Dose, Variable | Combination | Computed |
|---|---|---|---|
| Compound G (μM) | Gemcitabine (μM) | Fa | CI value* |
| 0.831 | 0.008 | 0.993 | 0.981 |
| 0.831 | 0.005926 | 0.989 | 0.826 |
| 0.831 | 0.00439 | 0.969 | 0.825 |
| 0.831 | 0.003252 | 0.93 | 0.793 |
| 0.831 | 0.002409 | 0.787 | 0.962 |
| 0.831 | 0.001784 | 0.66 | 1.094 |
| 0.831 | 0.001322 | 0.599 | 1.117 |

TABLE 6
Representative Compound B and Gemcitabine concentrations and
corresponding combination Fa and CI scores in MIAPACA2T2 cells.
MIAPACA2T2 Combination Index (CI) Determination

| Dose, Fixed | Dose, Variable | Combination | Computed |
|---|---|---|---|
| Compound B (μM) | Gemcitabine (μM) | Fa | CI value* |
| 0.2916 | 0.011 | 0.985 | 1.007 |
| 0.2916 | 0.007857 | 0.97 | 0.895 |
| 0.2916 | 0.005612 | 0.931 | 0.843 |
| 0.2916 | 0.004009 | 0.824 | 0.866 |
| 0.2916 | 0.002863 | 0.711 | 0.823 |
| 0.2916 | 0.002045 | 0.514 | 1.006 |
| 0.2916 | 0.001461 | 0.567 | 0.7 |

TABLE 7
Representative Compound G and Gemcitabine concentrations and
corresponding combination Fa and CI scores in PSN1 cells.
PSN1 Combination Index (CI) Determination

| Dose, Fixed | Dose, Variable | Combination | Computed |
|---|---|---|---|
| Compound G (μM) | Gemcitabine (μM) | Fa | CI value* |
| 0.0404 | 0.005 | 0.976 | 1.397 |
| 0.0404 | 0.003571 | 0.967 | 1.122 |
| 0.0404 | 0.002551 | 0.933 | 1.051 |
| 0.0404 | 0.001822 | 0.86 | 1.064 |
| 0.0404 | 0.001302 | 0.657 | 1.393 |
| 0.0404 | 0.00093 | 0.554 | 1.454 |
| 0.0404 | 0.000664 | 0.431 | 1.706 |

TABLE 8
Representative Compound B and Gemcitabine concentrations and
corresponding combination Fa and CI scores in PSN1 cells.
PSN1 Combination Index (CI) Determination

| Dose, Fixed | Dose, Variable | Combination | Computed |
|---|---|---|---|
| Compound B (μM) | Gemcitabine (μM) | Fa | CI value* |
| 0.022375 | 0.011 | 0.989 | 1.337 |
| 0.022375 | 0.007857 | 0.986 | 1.079 |
| 0.022375 | 0.005612 | 0.985 | 0.8 |
| 0.022375 | 0.004009 | 0.965 | 0.886 |
| 0.022375 | 0.002863 | 0.937 | 0.881 |
| 0.022375 | 0.002045 | 0.836 | 1.177 |
| 0.022375 | 0.001461 | 0.594 | 2.081 |

As shown in Tables 5-8, most of the CI scores are in the moderate synergy range.

### Example 6—Combination of PRMT5 Inhibitor and Gemcitabine in Pancreatic Cell Lines

Pancreatic cancer cell lines (MIAPACA2T2, PSN1) are treated with the combination of a PRMT5 inhibitor (i.e., Compound A) and gemcitabine for 6 days. PRMT5 inhibitor (i.e., Compound A) is performed at a 1.9-fold dilution series and the combination partner is performed at 1.2 to 1.7 fold dilution series to create an 8×10 dose matrix including DMSO-only controls. Cell viability is measured by the CellTiter-Glo Luminescence assay.

### Example 7—Combination of PRMT5 Inhibitor and Carboplatin in Pancreatic Cell Lines

Pancreatic cancer cell lines (MIAPACA2T2, PSN1) were treated with the combination of a PRMT5 inhibitor (i.e., Compound B or Compound G) and Carboplatin for 6 days. A PRMT5 inhibitor (i.e., Compound B or Compound G) was performed at a 1.9-fold dilution series and the combination partner was performed at 1.2 to 1.7 fold dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{T r e a t m e n t}{Average of vehicle} )$$

Synergy analysis was performed using the CalcuSyn software to determine CI scores based on the drug concentrations used and corresponding Fa values. Results are shown Tables 9-12 below. *CI Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1.

TABLE 9
Representative Compound G and Carboplatin concentrations and
corresponding combination Fa and CI scores in MIAPACA2T2 cells.
MIAPACA2T2 Combination Index (CI) Determination

| Dose, Fixed | Dose, Variable | Combination | Computed |
|---|---|---|---|
| Compound G (μM) | Carboplatin (μM) | Fa | CI value* |
| 0.831 | 15 | 0.966 | 0.396 |
| 0.831 | 11.1111 | 0.963 | 0.313 |
| 0.831 | 8.23045 | 0.939 | 0.322 |
| 0.831 | 6.09663 | 0.881 | 0.402 |
| 0.831 | 4.51602 | 0.832 | 0.437 |
| 0.831 | 3.3452 | 0.766 | 0.521 |
| 0.831 | 2.47793 | 0.711 | 0.599 |

TABLE 10
Representative Compound B and Carboplatin concentrations and
corresponding combination Fa and CI scores in MIAPACA2T2 cells.
MIAPACA2T2 Combination Index (CI) Determination

| Dose, Fixed | Dose, Variable | Combination | Computed |
|---|---|---|---|
| Compound B (μM) | Carboplatin (μM) | Fa | CI value* |
| 0.4374 | 15 | 0.943 | 0.32 |
| 0.4374 | 11.1111 | 0.932 | 0.269 |
| 0.4374 | 8.23045 | 0.869 | 0.326 |
| 0.4374 | 6.09663 | 0.82 | 0.331 |
| 0.4374 | 4.51602 | 0.771 | 0.339 |
| 0.4374 | 3.3452 | 0.712 | 0.377 |
| 0.4374 | 2.47793 | 0.653 | 0.442 |

TABLE 11
Representative Compound G and Carboplatin concentrations
and corresponding combination Fa and CI scores in PSN1 cells.
PSN1 Combination Index (CI) Determination

| Dose, Fixed | Dose, Variable | Combination | Computed |
|---|---|---|---|
| Compound G (μM) | Carboplatin (μM) | Fa | CI value* |
| 0.0404 | 3 | 0.9676 | 0.739 |
| 0.0404 | 2.14286 | 0.9439 | 0.726 |
| 0.0404 | 1.53061 | 0.9146 | 0.696 |
| 0.0404 | 1.09329 | 0.879 | 0.678 |
| 0.0404 | 0.780925 | 0.7473 | 0.997 |
| 0.0404 | 0.557803 | 0.7022 | 1.013 |
| 0.0404 | 0.398431 | 0.6699 | 1.016 |

TABLE 12
Representative Compound B and Carboplatin concentrations
and corresponding combination Fa and CI scores in PSN1 cells.
PSN1 Combination Index (CI) Determination

| Dose, Fixed | Dose, Variable | Combination | Computed |
|---|---|---|---|
| Compound B (μM) | Carboplatin (μM) | Fa | CI value* |
| 0.0384 | 3 | 0.957 | 0.547 |
| 0.0384 | 2.14286 | 0.878 | 0.813 |
| 0.0384 | 1.53061 | 0.817 | 0.919 |
| 0.0384 | 1.09329 | 0.701 | 1.259 |
| 0.0384 | 0.780925 | 0.648 | 1.376 |
| 0.0384 | 0.557803 | 0.671 | 1.187 |
| 0.0384 | 0.398431 | 0.688 | 1.057 |

As shown in Tables 10-12, most of the CI scores are in the synergy range.

### Example 8—Combination of PRMT5 Inhibitor and Carboplatin in Pancreatic Cell Lines

Pancreatic cancer cell lines (MIAPACA2T2, PSN1) are treated with the combination of a PRMT5 inhibitor (i.e., Compound A) and Carboplatin for 6 days. A PRMT5 inhibitor (i.e., Compound A) is performed at a 1.9-fold dilution series and the combination partner is performed at 1.2 to 1.7 fold dilution series to create an 8×10 dose matrix including DMSO-only controls. Cell viability is measured by the CellTiter-Glo Luminescence assay.

### Example 9—Combination of PRMT5 Inhibitor and Pemetrexed in NSCLC Cell Lines

NSCLC cancer cell lines (H292, A549) were treated with the combination of a PRMT5 inhibitor (i.e., Compound B or Compound G) and pemetrexed for 6 days. A PRMT5 inhibitor (i.e., Compound B or Compound G) was performed at a 1.9-fold dilution series and the combination partner was performed at 1.2 to 1.7 fold dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{T r e a t m e n t}{Average of vehicle} )$$

Synergy analysis was performed using the CalcuSyn software to determine CI scores based on the drug concentrations used and corresponding Fa values. Results are shown Tables 13-16 below. *CI Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1.

TABLE 13
Representative Compound G and Pemetrexed concentrations
and corresponding combination Fa and CI scores in H292 cells.
H292 Combination Index (CI) Determination

| Dose, Fixed | Dose, Variable | Combination | Computed |
|---|---|---|---|
| Compound G (μM) | Pemetrexed (μM) | Fa | CI value* |
| 0.437382 | 2 | 0.904 | 0.235 |
| 0.437382 | 1.17647 | 0.885 | 0.216 |
| 0.437382 | 0.692042 | 0.827 | 0.342 |
| 0.437382 | 0.407083 | 0.741 | 0.64 |
| 0.437382 | 0.239461 | 0.678 | 0.914 |
| 0.437382 | 0.140859 | 0.63 | 1.184 |
| 0.437382 | 0.082858 | 0.602 | 1.359 |

TABLE 14
Representative Compound B and Pemetrexed concentrations
and corresponding combination Fa and CI scores in H292 cells.
H292 Combination Index (CI) Determination

| Dose, Fixed | Dose, Variable | Combination | Computed |
|---|---|---|---|
| Compound B (μM) | Pemetrexed (μM) | Fa | CI value* |
| 0.1535 | 1.5 | 0.8508 | 0.385 |
| 0.1535 | 1 | 0.8703 | 0.216 |
| 0.1535 | 0.666667 | 0.8438 | 0.215 |
| 0.1535 | 0.444444 | 0.7887 | 0.309 |
| 0.1535 | 0.296296 | 0.7484 | 0.401 |
| 0.1535 | 0.197531 | 0.7032 | 0.587 |
| 0.1535 | 0.131687 | 0.6769 | 0.73 |

TABLE 15
Representative Compound G and Pemetrexed concentrations
and corresponding combination Fa and CI scores in A549 cells.
A549 Combination Index (CI) Determination

| Dose, Fixed | Dose, Variable | Combination | Computed |
|---|---|---|---|
| Compound G (μM) | Pemetrexed (μM) | Fa | CI value* |
| 0.437382 | 2 | 0.948 | 0.087 |
| 0.437382 | 1.17647 | 0.918 | 0.132 |
| 0.437382 | 0.692042 | 0.834 | 0.363 |
| 0.437382 | 0.407083 | 0.712 | 0.922 |
| 0.437382 | 0.239461 | 0.693 | 0.882 |
| 0.437382 | 0.140859 | 0.651 | 1.064 |
| 0.437382 | 0.082858 | 0.669 | 0.846 |

TABLE 16
Representative Compound B and Pemetrexed concentrations
and corresponding combination Fa and CI scores in A549 cells.
A549 Combination Index (CI) Determination

| Dose, Fixed | Dose, Variable | Combination | Computed |
|---|---|---|---|
| Compound B (μM) | Pemetrexed (μM) | Fa | CI value* |
| 0.1535 | 1 | 0.8717 | 0.139 |
| 0.1535 | 0.666667 | 0.8303 | 0.235 |
| 0.1535 | 0.444444 | 0.7384 | 0.639 |
| 0.1535 | 0.296296 | 0.6761 | 1.033 |
| 0.1535 | 0.197531 | 0.6484 | 1.172 |
| 0.1535 | 0.131687 | 0.6629 | 0.936 |
| 0.1535 | 0.087792 | 0.6401 | 1.06 |

As shown in Tables 13-16, most of the CI scores are in the synergy range.

### Example 10—Combination of PRMT5 Inhibitor and Pemetrexed in NSCLC Cell Lines

NSCLC cancer cell lines (H292) are treated with the combination of a PRMT5 inhibitor (i.e., Compound A) and pemetrexed for 6 days. A PRMT5 inhibitor (i.e., Compound A) is performed at a 1.9-fold dilution series and the combination partner is performed at 1.2 to 1.7 fold dilution series to create an 8×10 dose matrix including DMSO-only controls. Cell viability is measured by the CellTiter-Glo Luminescence assay.

### Example 11—Combination of PRMT5 Inhibitor and Irinotecan in Pancreatic Cell Lines

Pancreatic cancer cell lines (MIAPACA2T2, PSN1) were treated with the combination of PRMT5 inhibitor (i.e., Compound B or Compound G) and Irinotecan for 6 days. PRMT5 inhibitor (i.e., Compound B or Compound G) was performed at a 1.9-fold dilution series and the combination partner was performed at 1.2 to 1.7 fold dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{T r e a t m e n t}{Average of vehicle} )$$

Synergy analysis was performed using the CalcuSyn software to determine CI scores based on the drug concentrations used and corresponding Fa values. Results are shown Tables 17-20 below. *CI Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1.

TABLE 17
Representative Compound G and Irinotecan concentrations and
corresponding combination Fa and CI scores in MIAPACA2T2 cells.
MIAPACA2T2 Combination Index (CI) Determination

| Dose, Fixed | Dose, Variable | Combination | Computed |
|---|---|---|---|
| Compound G (μM) | Irinotecan (μM) | Fa | CI value* |
| 0.83102 | 0.5 | 0.9854 | 0.649 |
| 0.83102 | 0.4 | 0.9677 | 0.655 |
| 0.83102 | 0.32 | 0.9132 | 0.729 |
| 0.83102 | 0.256 | 0.8794 | 0.677 |
| 0.83102 | 0.2048 | 0.8028 | 0.72 |
| 0.83102 | 0.16384 | 0.7359 | 0.751 |
| 0.83102 | 0.131072 | 0.718 | 0.691 |

TABLE 18
Representative Compound B and Irinotecan concentrations and
corresponding combination Fa and CI scores in MIAPAC2T2 cells.
MIAPACA2T2 Combination Index (CI) Determination

| Dose, Fixed | Dose, Variable | Combination | Computed |
|---|---|---|---|
| Compound B (μM) | Irinotecan (μM) | Fa | CI value* |
| 0.4374 | 0.5 | 0.982 | 0.682 |
| 0.4374 | 0.4 | 0.962 | 0.672 |
| 0.4374 | 0.32 | 0.928 | 0.647 |
| 0.4374 | 0.256 | 0.885 | 0.602 |
| 0.4374 | 0.2048 | 0.827 | 0.571 |
| 0.4374 | 0.16384 | 0.774 | 0.545 |
| 0.4374 | 0.131072 | 0.731 | 0.531 |

TABLE 19
Representative Compound G and Irinotecan concentrations and
corresponding combination Fa and CI scores in PSN1 cells.
PSN1 Combination Index (CI) Determination

| Dose, Fixed | Dose, Variable | Combination | Computed |
|---|---|---|---|
| Compound G (μM) | Irinotecan (μM) | Fa | CI value* |
| 0.0213 | 0.4 | 0.9731 | 0.916 |
| 0.0213 | 0.266667 | 0.9402 | 0.836 |
| 0.0213 | 0.177778 | 0.8505 | 0.852 |
| 0.0213 | 0.118519 | 0.6137 | 1.087 |
| 0.0213 | 0.079012 | 0.4649 | 1.168 |
| 0.0213 | 0.052675 | 0.3585 | 1.28 |
| 0.0213 | 0.035117 | 0.4007 | 0.981 |

TABLE 20
Representative Compound B and Irinotecan concentrations and
corresponding combination Fa and CI scores in PSN1 cells.
PSN1 Combination Index (CI) Determination

| Dose, Fixed | Dose, Variable | Combination | Computed |
|---|---|---|---|
| Compound B (μM) | Irinotecan (μM) | Fa | CI value* |
| 0.0202 | 0.3 | 0.9634 | 0.956 |
| 0.0202 | 0.2 | 0.9412 | 0.8 |
| 0.0202 | 0.133333 | 0.8783 | 0.8 |
| 0.0202 | 0.088889 | 0.7988 | 0.806 |
| 0.0202 | 0.059259 | 0.7542 | 0.744 |
| 0.0202 | 0.039506 | 0.6339 | 0.899 |
| 0.0202 | 0.026337 | 0.4601 | 1.319 |

As shown in Tables 17-20, most of the CI scores are in the synergy range for MIAPACA2T2 and moderate synergy range for PSN1.

### Example 12—Combination of PRMT5 Inhibitor and Irinotecan in Pancreatic Cell Lines

Pancreatic cancer cell lines (MIAPACA2T2, PSN1) are treated with the combination of a PRMT5 inhibitor (i.e., Compound A) and Irinotecan for 6 days. PRMT5 inhibitor (i.e., Compound A) is performed at a 1.9-fold dilution series and the combination partner is performed at 1.2 to 1.7 fold dilution series to create an 8×10 dose matrix including DMSO-only controls. Cell viability is measured by the CellTiter-Glo Luminescence assay.

### Example 13—Combination of PRMT5 Inhibitor and 5-Fluoracil (5-FU) in Pancreatic Cell Lines

Pancreatic cancer cell lines (MIAPACA2T2, PSN1) were treated with the combination of a PRMT5 inhibitor (i.e., Compound B or Compound G) and 5-FU for 6 days. PRMT5 inhibitor (i.e., Compound B or Compound G) was performed at a 1.9-fold dilution series and the combination partner was performed at 1.2 to 1.7 fold dilution series to create an 8×10 dose matrix including DMSO-only controls. Cell viability was measured by the CellTiter-Glo Luminescence assay. Raw luminescent values were converted to fraction affected (Fa) with the following equation:

$$Fa = 1 - ( \frac{T r e a t m e n t}{Average of vehicle} )$$

Synergy analysis was performed using the CalcuSyn software to determine CI scores based on the drug concentrations used and corresponding Fa values. Results are shown Tables 21-24 below. *C Values (Calcusyn): Strong Synergism: 0.1-0.3; Synergism: 0.3-0.7; Moderate Synergism: 0.7-0.85; Slight Synergism: 0.85-0.9; Nearly Additive: 0.9-1.1.

TABLE 21
Representative Compound G and 5-FU concentrations and
corresponding combination Fa and CI scores in MIAPACA2T2 cells.
CalcuSyn software was used to generate CI scores.
MIAPACA2T2 Combination Index (CI) Determination

| Dose, Fixed | Dose, Variable | Combination | Computed |
|---|---|---|---|
| Compound G (μM) | 5-FU (μM) | Fa | CI value* |
| 0.83102 | 5 | 0.9246 | 0.834 |
| 0.83102 | 3.57143 | 0.9193 | 0.632 |
| 0.83102 | 2.55102 | 0.8941 | 0.555 |
| 0.83102 | 1.82216 | 0.85 | 0.529 |
| 0.83102 | 1.30154 | 0.8099 | 0.482 |
| 0.83102 | 0.929672 | 0.7511 | 0.477 |
| 0.83102 | 0.664052 | 0.7114 | 0.445 |

TABLE 22
Representative Compound B and 5-FU concentrations and
corresponding combination Fa and CI scores in MIAPACA2T2 cells.
MIAPACA2T2 Combination Index (CI) Determination

| Dose, Fixed | Dose, Variable | Combination | Computed |
|---|---|---|---|
| Compound B (μM) | 5-FU (μM) | Fa | CI value* |
| 0.4374 | 5 | 0.918 | 0.704 |
| 0.4374 | 3.57143 | 0.911 | 0.54 |
| 0.4374 | 2.55102 | 0.885 | 0.48 |
| 0.4374 | 1.82216 | 0.856 | 0.428 |
| 0.4374 | 1.30154 | 0.818 | 0.404 |
| 0.4374 | 0.929672 | 0.799 | 0.347 |
| 0.4374 | 0.664052 | 0.746 | 0.386 |

TABLE 23
Representative Compound G and 5-FU concentrations and
corresponding combination Fa and CI scores in PSN1 cells.
PSN1 Combination Index (CI) Determination

| Dose, Fixed | Dose, Variable | Combination | Computed |
|---|---|---|---|
| Compound G (μM) | 5-FU (μM) | Fa | CI value* |
| 0.0213 | 6 | 0.869 | 2.903 |
| 0.0213 | 3.75 | 0.836 | 2.158 |
| 0.0213 | 2.34375 | 0.789 | 1.668 |
| 0.0213 | 1.46484 | 0.74 | 1.282 |
| 0.0213 | 0.915527 | 0.669 | 1.055 |
| 0.0213 | 0.572205 | 0.416 | 1.404 |
| 0.0213 | 0.357628 | 0.3 | 1.422 |

TABLE 24
Representative Compound B and 5-FU concentrations and
corresponding combination Fa and CI scores in PSN1 cells.
PSN1 Combination Index (CI) Determination

| Dose, Fixed | Dose, Variable | Combination | Computed |
|---|---|---|---|
| Compound B (μM) | 5-FU (μM) | Fa | CI value* |
| 0.0202 | 6 | 0.9075 | 0.674 |
| 0.0202 | 3.75 | 0.8588 | 0.717 |
| 0.0202 | 2.34375 | 0.7273 | 1.056 |
| 0.0202 | 1.46484 | 0.6551 | 1.047 |
| 0.0202 | 0.915527 | 0.5174 | 1.305 |
| 0.0202 | 0.572205 | 0.453 | 1.299 |
| 0.0202 | 0.357628 | 0.5061 | 0.932 |

As shown in Tables 21-24, most of the CI scores are in the synergy range for MIAPACA2T2 and additive range for PSN1.

### Example 14—Combination of PRMT5 Inhibitor and 5-Fluoracil (5-FU) in Pancreatic Cell Lines

Pancreatic cancer cell lines (MIAPACA2T2, PSN1) are treated with the combination of a PRMT5 inhibitor (i.e., Compound A) and 5-EU for 6 days. PRMT5 inhibitor (i.e., Compound A) is performed at a 1.9-fold dilution series and the combination partner is performed at 1.2 to 1.7 fold dilution series to create an 8×10 dose matrix including DMSO-only controls. Cell viability is measured by the CellTiter-Glo Luminescence assay.

### Example 15—PRMT5 Inhibitor Inhibited Growth in Multiple MTAP-Null Tumor Xenograft Models

6 Female NOD/SCID mice were implanted with patient-derived tumor xenograft (PDX) models of pancreatic ovarian, esophageal, melanoma, lung, brain, mixed mullerian or gallbladder cancer. Mean tumor volumes for each grouping were between 100-200 mm³. Mice were allocated to the 2 different study groups by tumor volume and dosing was initiated with Vehicle or Compound B at 100 mg/kg orally once daily. Plotted data represents the TGI (tumor growth inhibition), n=3 for each group.

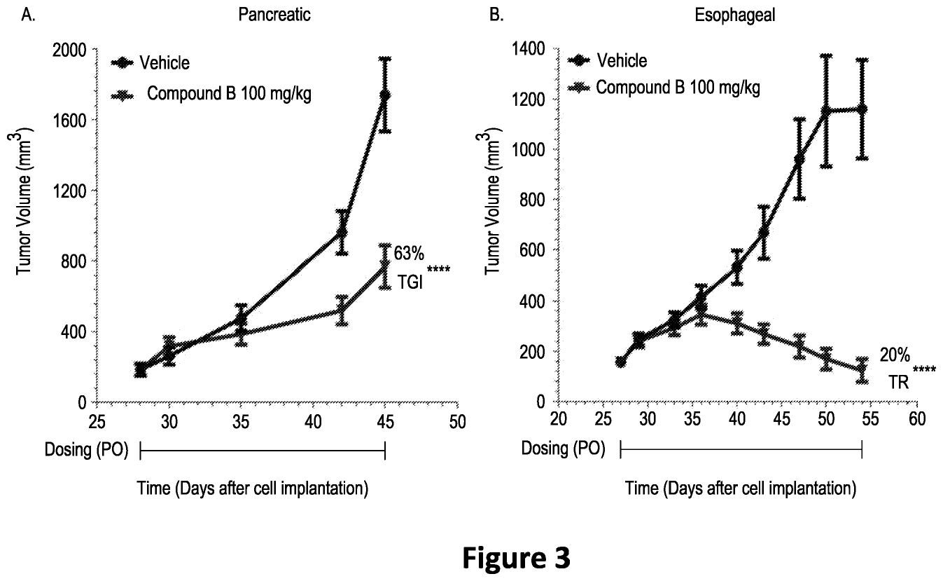

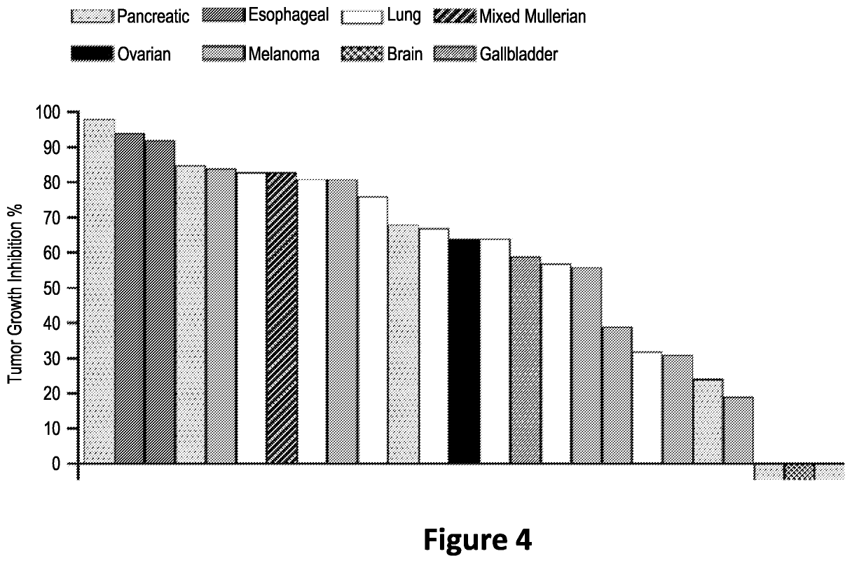

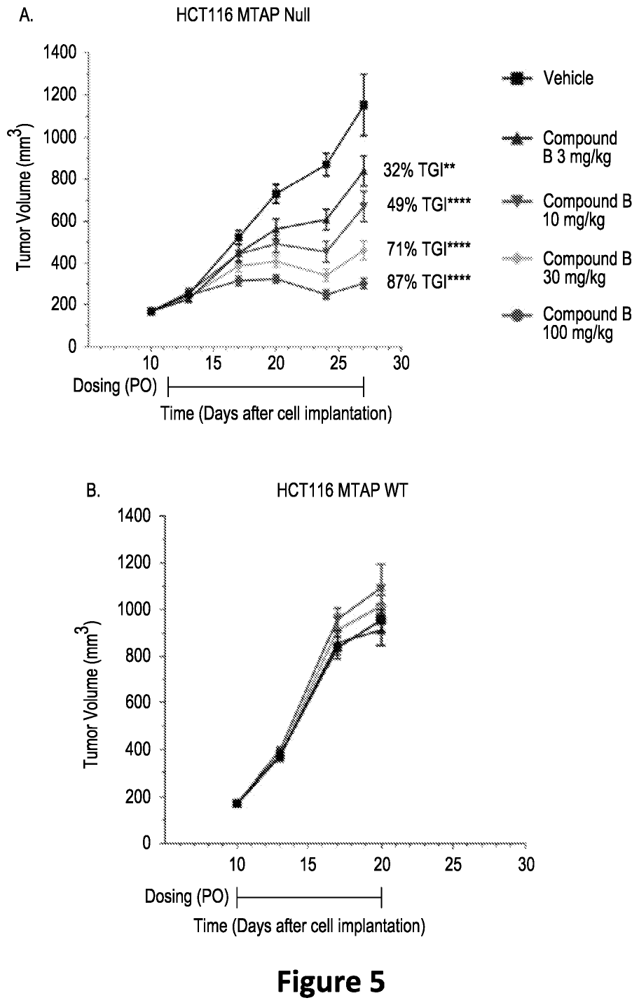

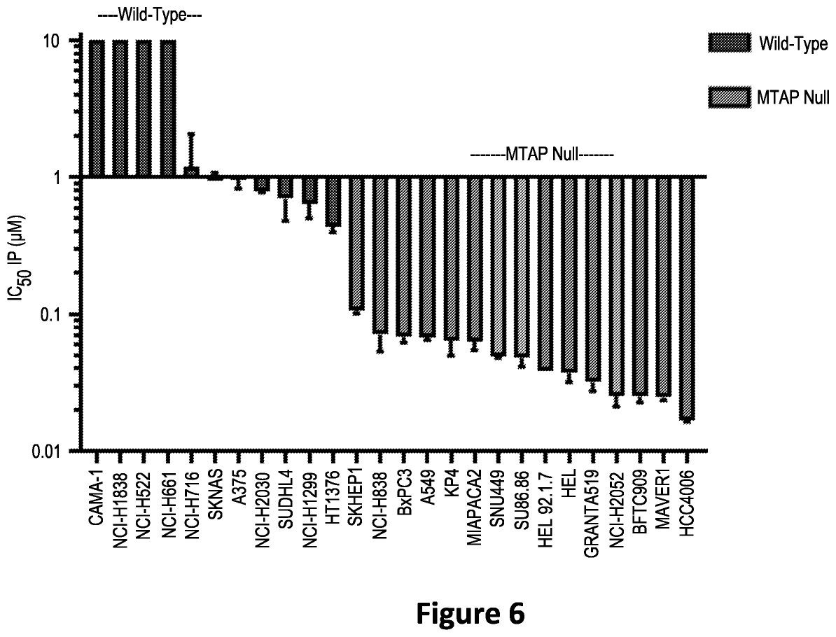

Furthermore, treatment with Compound B inhibits the growth of multiple MTAP-null tumor xenograft models, BXPC3 (PDAC) and DOHH2 (DLBCL) (FIG. 3). Compound B was profiled against a panel of over twenty PDX models (FIG. 6), with greater than 50% tumor growth inhibition observed in the majority of PDX models harboring deletion of the MTAP gene (FIG. 4).

The data provided herein demonstrates that PRMT5 inhibitors that selectively target PRMT5 in cooperation with MTA may represent a novel and compelling therapeutic strategy for the treatment of MTAP-null cancers.

### Example 16—PRMT5 Inhibitor Inhibited Growth in Multiple MTAP-Null Tumor Xenograft Models

Female NOD/SCID mice are implanted with patient-derived tumor xenograft (PDX) models of pancreatic ovarian, esophageal, melanoma, lung, brain, mixed mullerian or gallbladder cancer. Mice are allocated to the 2 different study groups by tumor volume and dosing is initiated with Vehicle or Compound A at 100 mg/kg orally once daily. Tumor volume is assessed over time and plotted to provide tumor growth inhibition (TGI).

### Example 17—Combination of PRMT5 Inhibitor and Paclitaxel Inhibited Tumor Volume a NSCLC Animal Model

10 Female NOD/SCID mice were implanted with H292 NSCLC tumor xenografts. Mean tumor volumes for each grouping were between 100-200 mm³. Mice were allocated to the 2 different study groups by tumor volume and dosing was initiated with Vehicle or Compound G (100 mg/kg) or Compound B (100 mg/kg) orally once daily in combination with paclitaxel (20 mg/kg). Plotted data represents the TGI (tumor growth inhibition), n=10 for each group.

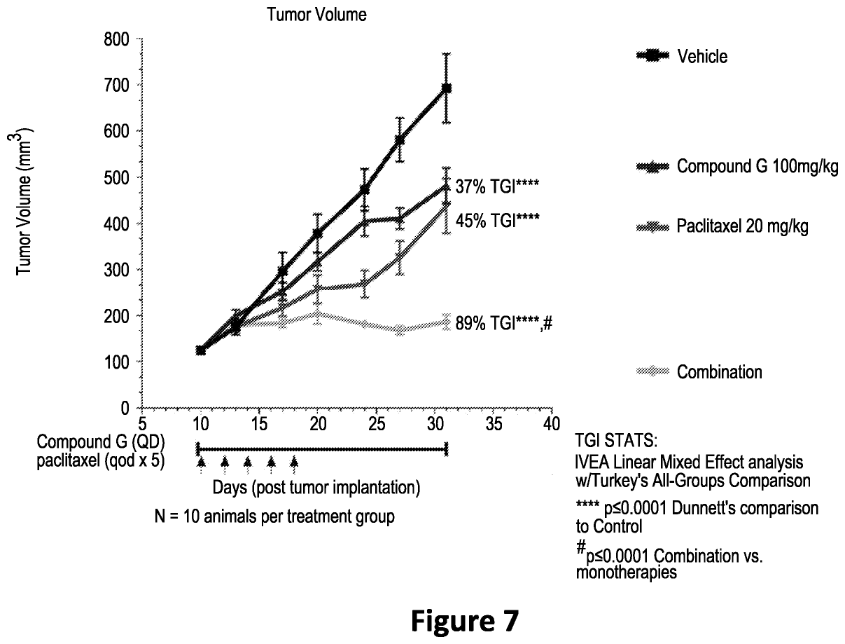

Results showed that the combination of Compound G and paclitaxel resulted in significant anti-tumor activity versus either single agent alone H292 NSCLC xenografts. See FIG. 7.

### Example 18—Combination of PRMT5 Inhibitor and Paclitaxel Inhibited Tumor Volume a NSCLC Animal Model

Female NOD/SCID mice are implanted with H292 NSCLC tumor xenografts. Mice are allocated to the 2 different study groups by tumor volume and dosing is initiated with Vehicle or Compound A (100 mg/kg) orally once daily in combination with paclitaxel (20 mg/kg). Tumor volume is assessed over time and plotted to provide tumor growth inhibition (TGI).

### Example 19—Combination of PRMT5 Inhibitor and Paclitaxel Inhibited Tumor Volume in a H292 NSCLC Animal Model

10 Female NOD/SCID mice were implanted with H292 tumor xenografts. Mean tumor volumes for each grouping were between 100-200 mm³. Mice were allocated to the 2 different study groups by tumor volume and dosing was initiated with Vehicle or Compound G (100 mg/kg) orally once daily in combination with paclitaxel (20 mg/kg). Paclitaxel was administered intraperitoneally (IP) starting on Day 10 and then dosed every other day for a total of five doses. Plotted data represents the TGI (tumor growth inhibition), n=10 for each group.

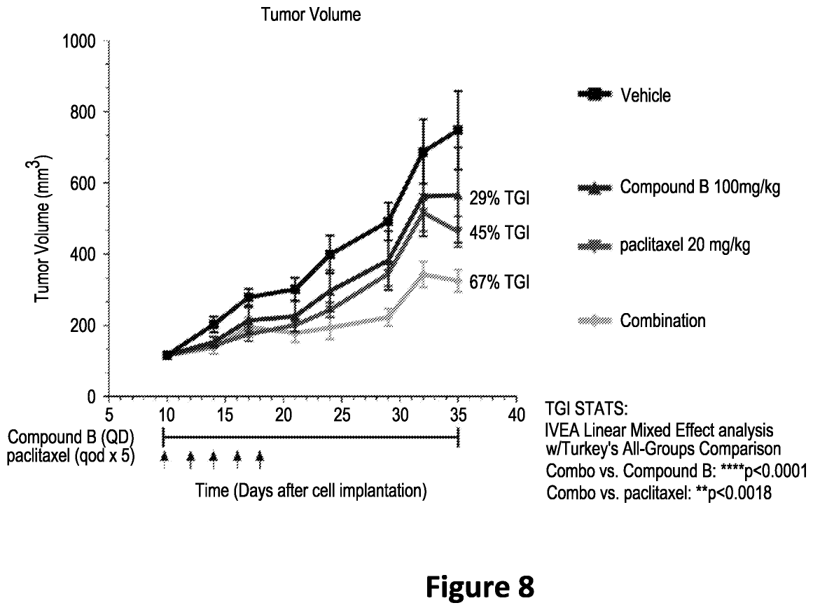

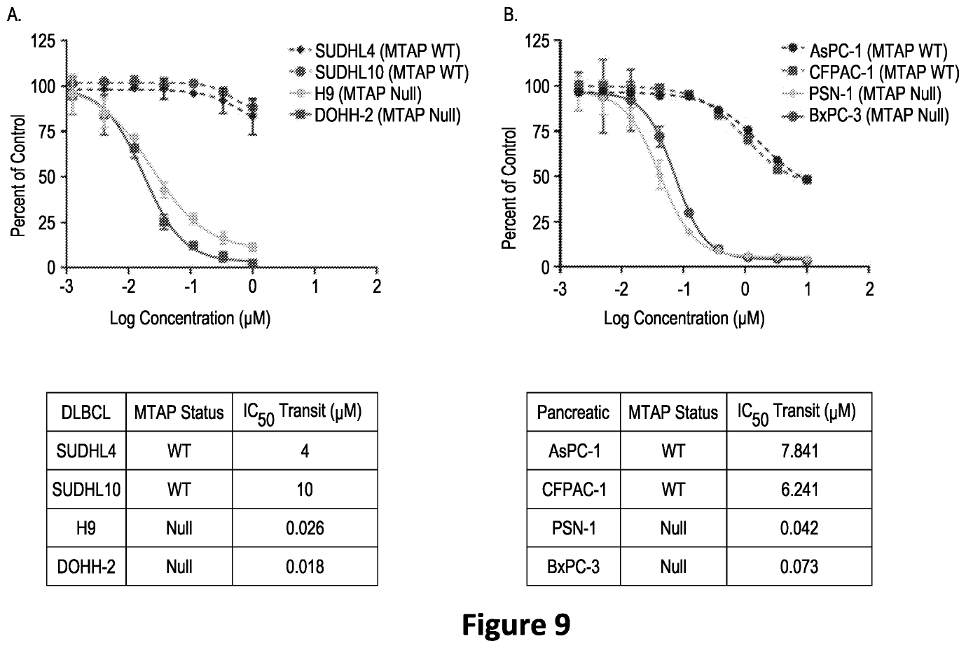

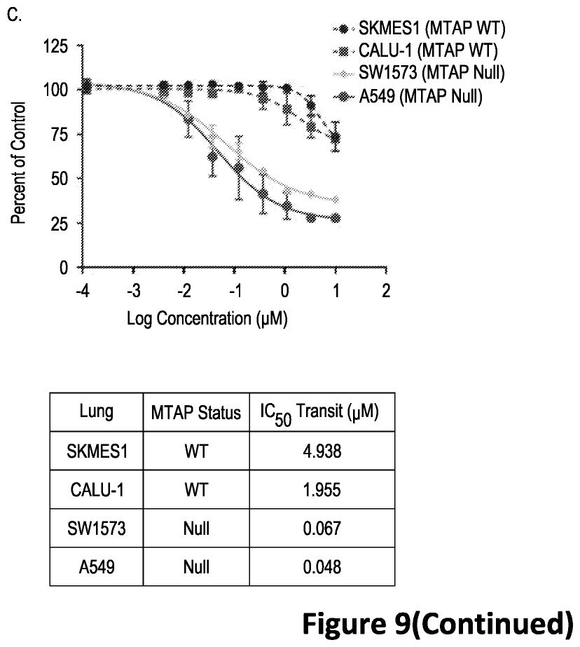

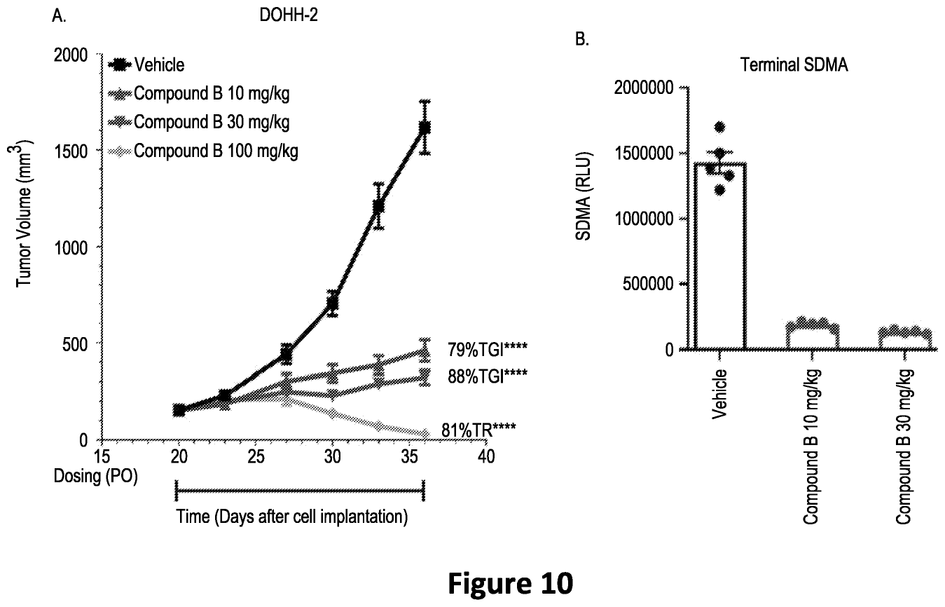

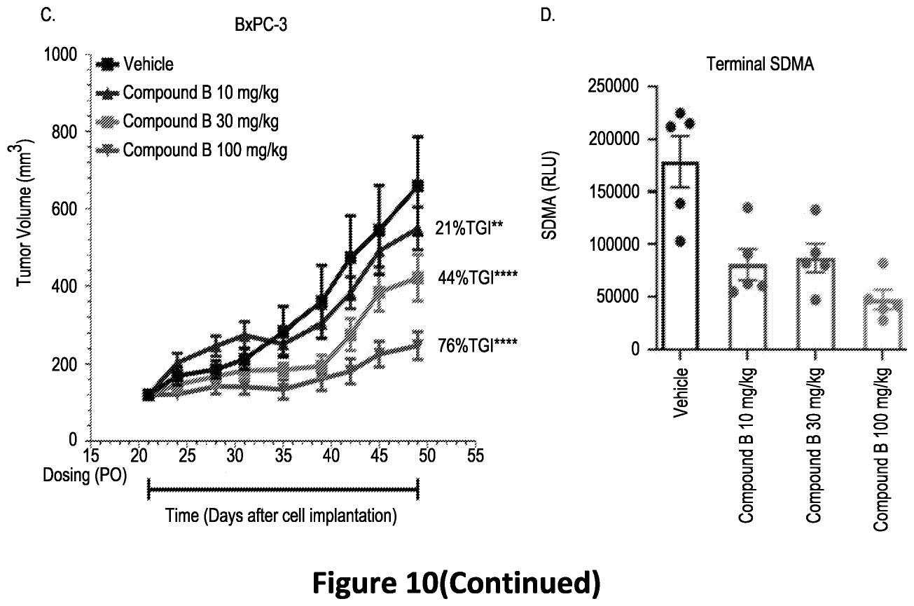

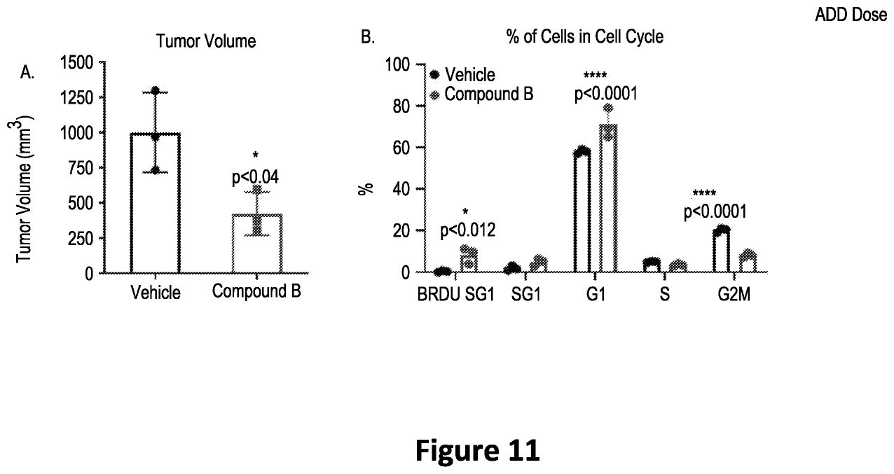

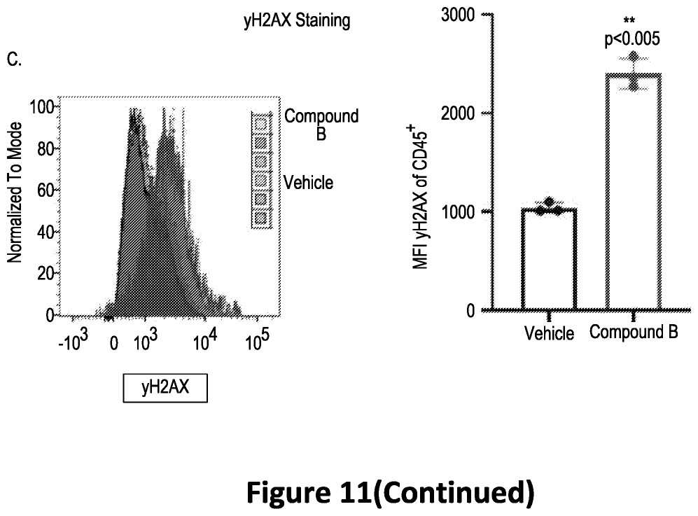

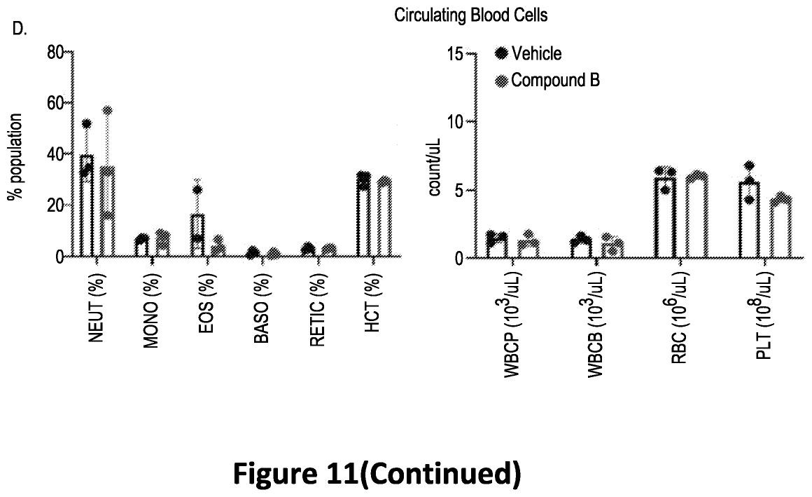

Results showed that the combination of Compound G and paclitaxel results in significant anti-tumor activity versus either single agent alone H292 NSCLC xenografts. See FIG. 7. Similarly, the combination of Compound B and paclitaxel results in significant anti-tumor activity versus either single agent alone H292 NSCLC xenografts. See FIG. 8.

### Example 20—Combination of PRMT5 Inhibitor and Paclitaxel Inhibited Tumor Volume in a H292 NSCLC Animal Model

Female NOD/SCID mice are implanted with H292 xenografts. Mice are allocated to the 2 different study groups by tumor volume and dosing is initiated with Vehicle or Compound A (100 mg/kg) orally once daily in combination with paclitaxel. Tumor volume is assessed over time and plotted to provide tumor growth inhibition (TGI).

### Example 21—PRMT5 Inhibitor Monotherapy in Adult Patients with Metastatic or Locally Advanced MTAP-Null Solid Tumors

The following study will be performed to evaluate the safety, tolerability, and to determine the maximum tolerated dose (MTD) or recommended phase 2 dose (RP2D) of PRMT5 inhibitor monotherapy in adult patients with metastatic or locally advanced MTAP-null solid tumors. Pharmacokinetics (PK) of the PRMT5 inhibitor monotherapy will be assessed. In addition, the following will be evaluated: objective response rate (ORR), disease control rate (DCR), duration of response (DoR), time to response (TTR), duration of stable disease (SD), progression-free survival (PFS), and overall survival (OS) of PRMT5 inhibitor in adult patients with MTAP-null solid tumors.

This study is conducted in 3 parts, each with subparts. Part 1a/b (dose exploration, 5 dose levels) will enroll approximately 30 patients from any eligible tumor type.

Treatment will continue until progression or withdrawal. Safety follow-up is approximately 30 (±3) days after the last dose of PRMT5 inhibitor, or before initiation of other therapy, whichever occurs first. Long-term follow-up is every 6 months for up to 2 years from the first dose for all patients who have not withdrawn consent.

Intra-patient dose escalations are allowed. Patients who complete the dose-limiting toxicity (DLT) period may proceed to a higher dose level, not exceeding the highest dose level deemed to be safe by the Dose Level Review Team (DLRT), provided that no DLT has been reported for the patient during or after completion of the DLT period, and the patient has not experienced any grade !2 adverse events (deemed treatment-related by the investigator) during treatment.

Dose exploration will estimate the MTD using a Bayesian logistic regression model (BLRM) design. At completion of each cohort, the DLRT will recommend the next dose level as follows: (1) dose level recommendation from the BLRM, and by evaluating available safety data, laboratory results, and PK information; (2) a RP2D may be identified based on emerging safety, efficacy, PK, and PD data before reaching an MTD.

A minimum of 6 DLT-evaluable patients are required to be treated at the monotherapy RP2D in this dose exploration part of the study, before proceeding to dose expansion.

Phase 1a/b study endpoints

| | Endpoint |
|---|---|
| Primary | DLTs, TEAEs, SAEs, changes in vital signs, |
| | ECGs, and clinical laboratory tests |
| Secondary | PK parameters of PRMT5 inhibitor, including Cmax, |
| | Tmax and AUC after single dose and multiple doses |

*DLTs= dose limiting toxicities;
TEAEs = treatment-emergent adverse events;
ECGs = echocardiograms

### Endpoint Definitions

Objective response (defined as the best overall response of confirmed complete response or partial response based on RECIST v1.1) derived utilizing investigator tumor assessments
Disease control is defined as confirmed objective response or stable disease (SD) based on RECIST v1.1.
Duration of response (DoR), based on RECIST v1.1, is defined as the time from the first documentation of objective response which is subsequently confirmed, until the first documentation of disease progression or death due to any cause, whichever occurs first.
Time to response is defined as the time from enrollment until the first documentation of objective response, which is subsequently confirmed, based on RECIST v1.1.
Duration of SD, based on RECIST v1.1, is defined as the time from first dose of PRMT5 inhibitor until the first documentation of radiographic disease progression or death due to any cause, whichever occurs first.
Progression-free survival (PFS), is defined as the time from the first dose of PRMT5 inhibitor until the first documentation of radiologic disease progression or death due to any cause, whichever occurs first in the absence of subsequent anticancer therapy. PFS will be censored at the last evaluable post-baseline tumor assessment prior to subsequent anticancer therapy; otherwise, at the first dose of PRMT5 inhibitor. Progression will be based on RECIST v1.1 derived utilizing investigator tumor assessments

Overall survival (OS) is defined as the time from the first dose of PRMT5 inhibitor until death due to any cause. OS is censored at the last date known to be alive through the data cutoff date.

### Example 22—Combination of a PRMT5 Inhibitor and Paclitaxel or Carboplatin in NSCLC Cell Lines

NSCLC cell lines (H292) are treated with the combination of a PRMT5 inhibitor (i.e., Compound B) and paclitaxel or carboplatin for 6 days. PRMT5 inhibitor (Compound B) is performed at a 1.9-fold dilution series and the combination partner is performed at 1.2 to 1.7 fold dilution series to create an 8×10 dose matrix including DMSO-only controls.

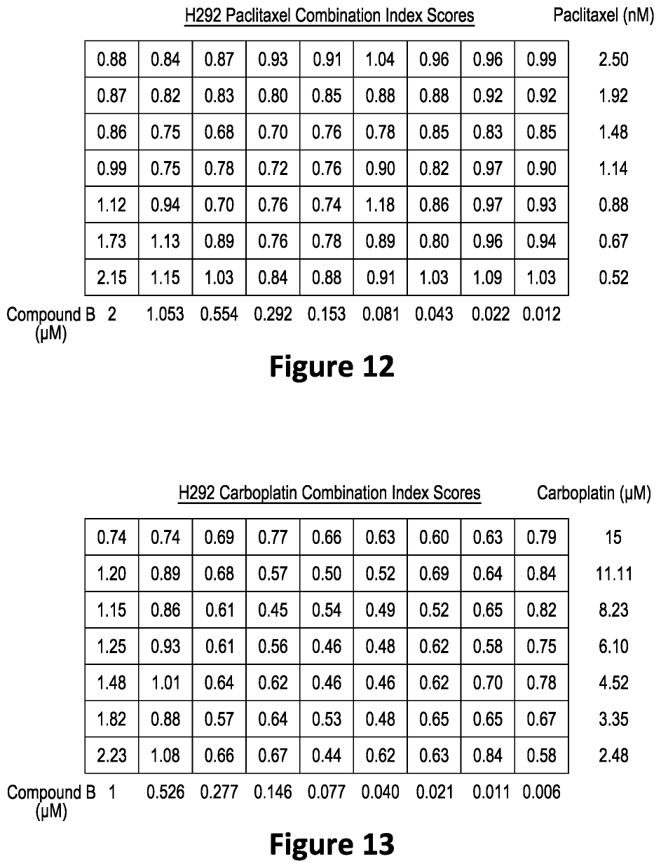

Synergy analysis was performed using the CalcuSyn software to determine Combination Index (CI) scores based on the drug concentrations used and corresponding Fa values. CI<1 indicated synergy. CI=1 indicates additivity. CI>1 indicates antagonism. Results are shown in FIGS. 12 and 13.

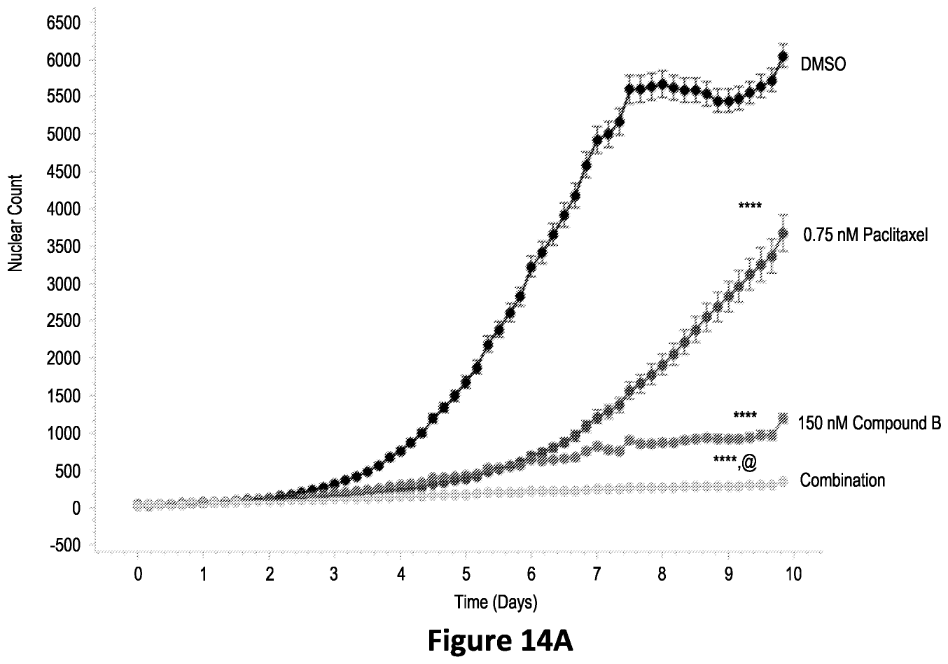

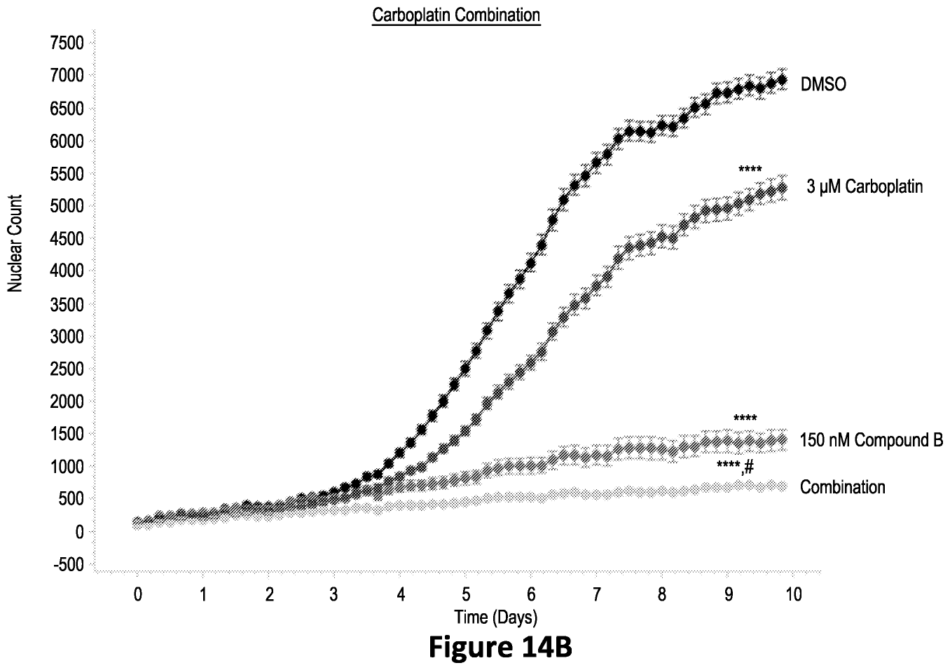

To assess cell growth after combination treatment, nuclear counts were performed on the IncuCyte live cell imager over 10 days. Results showed that the combination of Compound B and paclitaxel (FIG. 14A) and the combination of Compound B and carboplatin (FIG. 14B) results in significant anti-tumor cell growth activity versus either paclitaxel or carboplatin alone in a NSCLC (H292) cell line.

### Example 23—Combination of a PRMT5 Inhibitor and Paclitaxel or Carboplatin Inhibited Tumor Volume in a H292 MTAP Null NSCLC Xenografts

10 Female NOD/SCID mice were implanted with H292 tumor xenografts. Mean tumor volumes for each grouping were between 100-200 mm³. Mice were allocated to the 2 different study groups by tumor volume and dosing was initiated with Vehicle or B1 (00 mg/kg) orally once daily in combination with paclitaxel (20 mg/kg) carboplatin (60 mg/kg). Paclitaxel was administered intraperitoneally (IP) starting on Day 10 and then dosed every other day for a total of five doses. Carboplatin was administered intraperitoneally (IP) starting on Day 10 and then dosed 1×/week for a total of 3 doses. Plotted data represents the TGI (tumor growth inhibition), n=10 for each group.

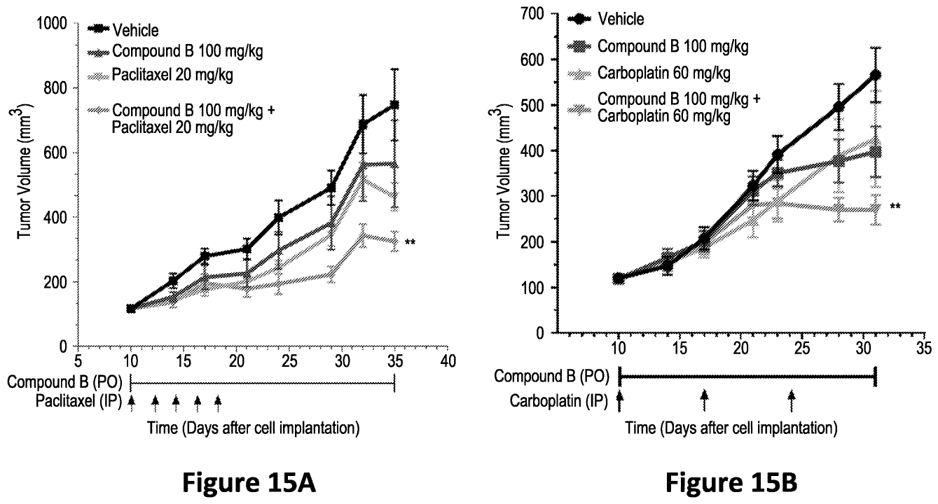

Results showed that the combination of Compound B and paclitaxel (FIG. 15A) and the combination of Compound B and carboplatin (FIG. 15B) results in significant anti-tumor activity versus paclitaxel or carboplatin alone in H292 NSCLC xenografts.

What is claimed is:

1. A method of treating cancer in a patient in need thereof comprising administering a PRMT5 inhibitor in an amount ranging from 40 mg to 2000 mg to the patient, wherein the PRMT5 inhibitor comprises a compound of <Formula 1> or Compound (B) or a pharmaceutically acceptable salt thereof: 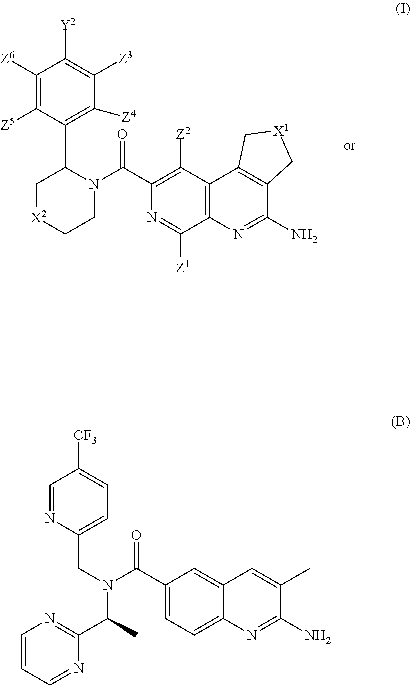
wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride.

2. The method of claim 1, wherein the cancer is a MTAP-null cancer, optionally wherein the MTAP-null cancer is glioblastoma, mesothelioma, soft tissue sarcoma, esophageal cancer, melanoma, lymphoma/leukemia, head and neck cancer, cholangiocarcinoma, stomach cancer, glioma, thymoma, adenocystic carcinoma, pancreatic cancer, lung cancer, breast cancer, liver cancer or bladder cancer.

3. The method of claim 2, wherein the lung cancer is non-squamous cell lung cancer (NSCLC).

4. The method of any one of claims 1-3, wherein the PRMT5 inhibitor has a structure of Formula (S)-I, or a pharmaceutically acceptable salt thereof:

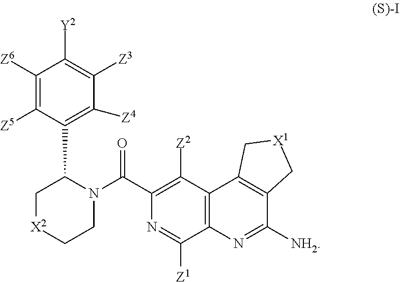

5. The method of any one of claims 1-4, wherein:
X¹ is O;
Z¹ and Z² are each H;
X² is O;
Z³, Z⁴, Z⁵, and Z⁶ are each H;
Y² is C₁-C₆haloalkyl; and
Y² is CF₃.

6. The method of any one of claims 1-5, further comprising administering paclitaxel to the subject, optionally administering to the subject (a) 40-2000 mg of the PRMT5 inhibitor; and (b) 75 mg/m²-135 mg/m² paclitaxel.

7. The method of any one of claims 1-5, further comprising administering carboplatin to the subject, optionally administering to the subject (a) 40-2000 mg of the PRMT5 inhibitor; and (b) AUC5-AUC6 carboplatin.

8. The method of any one of claims 1-5, further comprising administering gemcitabine to the subject, optionally administering to the subject (a) 40-2000 mg of the PRMT5 inhibitor; and (b) 1000 mg/m²-1250 mg/m² gemcitabine.

9. The method of any one of claims 1-5, further comprising administering irinotecan to the subject, optionally administering to the subject (a) 40-2000 mg of the PRMT5 inhibitor; and (b) 150 mg/m²-180 mg/m² irinotecan.

10. The method of any one of claims 1-5, further comprising administering 5-fluoracil to the subject, optionally administering to the subject (a) 40-2000 mg of the PRMT5 inhibitor; and (b) 400 mg/m²-1200 mg/m² 5-fluoracil.

11. The method of any one of claims 1-5, further comprising administering pemetrexed to the subject, optionally administering to the subject (a) 40-2000 mg of the PRMT5 inhibitor; and (b) 500 mg/m² pemetrexed.

12. The method of any one of claims 1-11, wherein the PRMT5 inhibitor comprises a compound having a structure of Compound G: 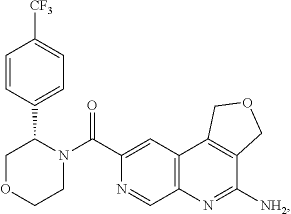 [SMILES: Nc1nc2cnc(C(=O)N3CCOC[C@@H]3c3ccc(C(F)(F)F)cc3)cc2c2c1COC2]
or salt thereof.

13. The method of any one of claims 1-11, wherein the PRMT5 inhibitor comprises Compound B or salt thereof.

14. The method of any one of claims 1-11, wherein the PRMT5 inhibitor comprises a compound having a structure of Compound A: 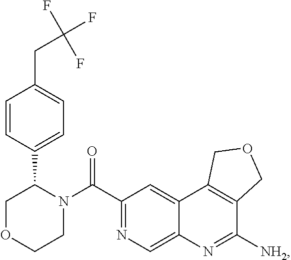 [SMILES: Nc1nc2cnc(C(=O)N3CCOC[C@@H]3c3ccc(CC(F)(F)F)cc3)cc2c2c1COC2]
or salt thereof.

15. A method of treating cancer in a patient in need thereof comprising administering to the patient
(a) a PRMT5 inhibitor in an amount ranging from 40 mg to 2000 mg, wherein the PRMT5 inhibitor comprises a compound set forth in <Formula 1> or Compound B, or a pharmaceutically acceptable salt thereof; 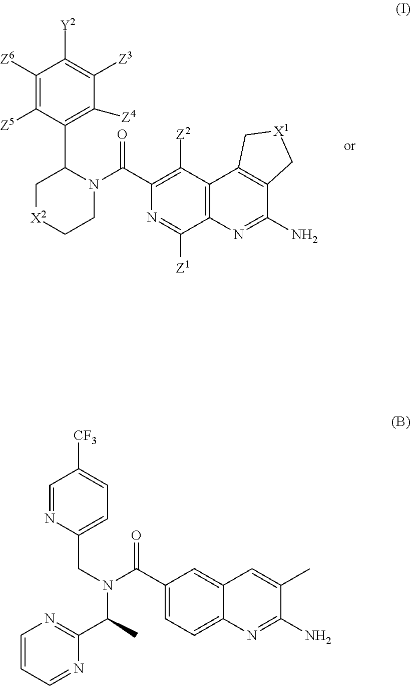
wherein
X¹ is NH, N(C₁-C₆alkyl), O, or S;
X² is N(C₁-C₆alkyl), O, or S;
Y² is H, C₁-C₆ alkyl, or C₁-C₆ haloalkyl;
each of Z¹ and Z² is independently H, F, or C₁-C₆ alkyl; and
each of Z³, Z⁴, Z⁵, and Z⁶ is independently H, C₁-C₆alkyl, or chloride; and
(b) a standard of care therapy for the treatment of cancer.

16. The method of claim 15, wherein the standard of care therapy comprises chemotherapy, optionally wherein the chemotherapy comprises paclitaxel, carboplatin, gemcitabine, irinotecan, 5-fluoracil or pemetrexed or a combination thereof.

17. The method of claim 15 or claim 16, wherein the PRMT5 inhibitor has a structure of Formula (S)-I, or a pharmaceutically acceptable salt thereof:

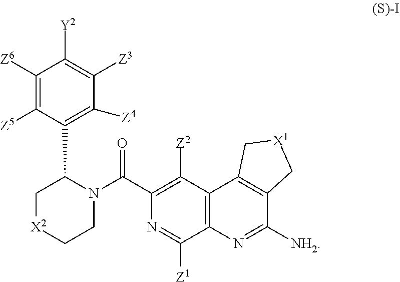

18. The method of any one of claims 15-17, wherein
X¹ is O;
Z¹ and Z² are each H;
X² is O;
Z³, Z⁴, Z⁵, and Z⁶ are each H;
Y² is C₁-C₆haloalkyl; and
Y² is CF₃.

19. The method of any one of claims 16-18 wherein the chemotherapy comprises paclitaxel, and the method optionally comprises administering to the subject (a) 40-2000 mg of the PRMT5 inhibitor; and (b) 75 mg/m²-135 mg/m² paclitaxel.

20. The method of any one of claims 16-18, wherein the chemotherapy comprises carboplatin, and the method optionally comprises administering to the subject (a) 40-2-00 mg of the PRMT5 inhibitor; and (b) AUC5-AUC6 carboplatin.

21. The method of any one of claims 16-18, wherein the chemotherapy comprises gemcitabine, and the method optionally comprises administering to the subject (a) 40-2000 mg of the PRMT5 inhibitor; and (b) 1000 mg/m²-1250 mg/m² gemcitabine.

22. The method of any one of claims 16-18, wherein the chemotherapy comprises irinotecan, and the method optionally comprises administering to the subject (a) 40-2000 mg of the PRMT5 inhibitor; and (b) 150 mg/m²-180 mg/m² irinotecan.

23. The method of any one of claims 16-18, wherein the chemotherapy comprises 5-fluoracil, and the method optionally comprises administering to the subject (a) 40-2000 mg of the PRMT5 inhibitor; and (b) 400 mg/m²-1200 mg/m² 5-fluoracil.

24. The method of any one of claims 16-18, wherein the chemotherapy comprises pemetrexed, and the method optionally comprises administering to the subject (a) 40-2000 mg of the PRMT5 inhibitor; and (b) 500 mg/m² pemetrexed.

25. The method of any one of claims 15-24, wherein the PRMT5 inhibitor comprises a compound having a structure of Compound G: 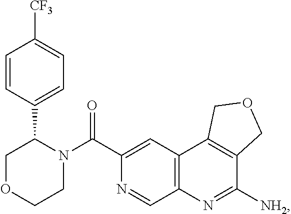 [SMILES: Nc1nc2cnc(C(=O)N3CCOC[C@@H]3c3ccc(C(F)(F)F)cc3)cc2c2c1COC2]
or salt thereof.

26. The method of any one of claims 15-24, wherein the PRMT5 inhibitor comprises Compound B, or salt thereof.

27. The method of any one of claims 15-24, wherein the PRMT5 inhibitor is a compound having a structure of Compound A: 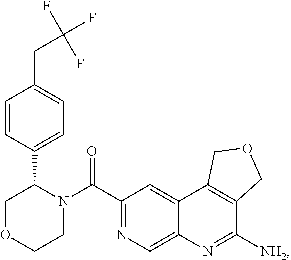 [SMILES: Nc1nc2cnc(C(=O)N3CCOC[C@@H]3c3ccc(CC(F)(F)F)cc3)cc2c2c1COC2]
or salt thereof.

28. The method of any one of claims 15-27, wherein the PRMT5 inhibitor and the standard of care therapy are administered concurrently.

29. The method of any one of claims 15-27, wherein the PRMT5 inhibitor and the standard of care therapy are administered sequentially.
